(19) United States
(12) Patent Application Publication
(10) Pub. No.: US 2019/0029756 A1
(43) Pub. Date: Jan. 31, 2019

(54) SYSTEM FOR OUT OF BORE FOCAL LASER THERAPY

(71) Applicant: THE REGENTS OF THE UNIVERSITY OF CALIFORNIA, Oakland, CA (US)

(72) Inventors: Shyam Natarajan, Santa Monica, CA (US); Alan Martin Priester, Westminster, CA (US); James Garritano, Los Angeles, CA (US); Leonard Marks, Beverly Hills, CA (US); Warren Grundfest, Los Angeles, CA (US); Rory Geoghegan, Los Angeles, CA (US)

(21) Appl. No.: 16/072,979

(22) PCT Filed: Jan. 26, 2017
(86) PCT No.: PCT/US17/15088

§ 371 Date: Jul. 26, 2018

Related U.S. Application Data
(60) Provisional application No. 62/287,105, filed on Jan. 26, 2016.

(51) Int. Cl. A61B 18/24; A61B 5/055; A61B 5/00; A61B 10/02; A61B 34/10; A61B 8/12; A61B 8/08

(52) U.S. Cl. CPC A61B 18/24; A61B 5/055; A61B 5/0036; A61B 2017/3405; A61B 34/10; A61B 8/12; A61B 8/08; A61B 10/0233

(57) ABSTRACT

The present invention provides methods of determining cancer margins indicating the location and breadth of treatment necessary for the elimination of cancerous tissue during focal laser therapy. The present invention also provides systems and devices for focal laser therapy, and methods for using the same. The present invention does not rely on MRI thermometry, improving accuracy of treatment while also reducing treatment time and cost.

### CROSS-REFERENCE TO RELATED APPLICATIONS

This application claims priority to U.S. Provisional Patent Application No. 62/287,105 filed Jan. 26, 2016, the contents of which are incorporated by reference herein in their entirety.

### BACKGROUND OF THE INVENTION

Prostate cancer (PCa) is the 5th most common cancer and the 2nd most common in men (Kamangar F et al., J. Clin. Oncol. 24 (2006):2137-2150). Traditionally PCa has been treated by either radical-whole gland therapy or active surveillance (AS) (Valerio M et al., Eur. Urol. 66.4 (2014): 732-751). Deciding the appropriate course of action is challenging as radical prostatectomy (RP) has been shown to significantly reduce mortality while many men under AS never require radical intervention (Bill-Axelson A et al., N. Engl. J. Med. 370 (2014):932-942). Despite being associated with numerous side effects including erectile dysfunction, urinary incontinence and rectal toxicity (Kasivisvanathan V et al., Clin. Oncol. 25, (2013):461-473), RP remains appropriate for those with high risk PCa (Heidenreich A et al., Eur. Urol. 59, (2011):61-71). AS is suitable for men with low-risk PCa (Tosoian J J et al., J. Clin. Oncol. 29, (2011):2185-2190; Bul M et al., Eur. Urol. 63, (2013):597-603); however, due to factors such as the fear of living with a potentially lethal condition more than 90% of eligible men elect for intervention over AS (Barocas D A et al., J. Urol. 180, (2008):1330-1335). In addition to the associated morbidity, the current trend of overtreatment of PCa has huge cost implications. A recent report found that ‘The ability to avoid treating the 80% of men with low-grade disease who will never die of prostate cancer would save $1.32 billion per year nationally’ (Aizer AA et al., J. Natl. Compr. Canc. Netw. 13, (2015):61-68). Given its inherently low level of associated complications and minimally invasive nature focal therapy may provide a low-cost alternative to traditional therapy for both low and intermediate risk PCa.

Laser interstitial thermal therapy (LITT) has been demonstrated as a safe and effective form of focal therapy for the treatment of PCa (Natarajan S et al., J. Urol. 196, (2015):1-8; Eggener SE et al., J. Urol. (2016):3-8; Oto A et al., MR Imaging—guided Focal Laser Ablation for Prostate Cancer : Phase 1 Trial. (2013):267). LITT consists of inserting a diffusing laser fiber into the target and raising tissue temperatures to 60-95° C. In order to achieve cancer control and prevent damage to surrounding structures this treatment modality requires real-time feedback of tissue coagulation. Tissue charring is reduced via an active cooling catheter that circulates saline around the laser fiber although other cooling methods such as Peltier coolers can be used. The standard approach of monitoring temperature during LITT is magnetic resonance thermometry (MRT), which is time-consuming, labor intensive and expensive. The critical barrier to the widespread adoption of LITT is its reliance on magnetic resonance thermometry (MRT) and temperature-time thermal dose models. Furthermore, the Arrhenius damage calculation, commonly used in tandem with focal laser therapy systems as an efficacy monitor, has thus far proven to be unreliable in determining the true extent of thermally induced tissue damage.

There is a need in the art for an improved system and method for focal laser therapy of soft tissue. The present invention meets this need.

### SUMMARY OF THE INVENTION

In one aspect, the present invention relates to a method of cancer margin determination in soft tissue. The method comprises the steps of acquiring at least one MRI image of a region of interest having at least one MRI-visible lesion; generating a 3D model of the at least one MRI-visible lesion from the at least one MRI image; acquiring at least one biopsy core from the tissue surrounding the MRI-visible lesion; categorizing the at least one biopsy core as a cancer-containing positive node, a cancer-absent negative node, or a neutral node having an indeterminate cancer presence; at least partially expanding the 3D model of the at least one MRI-visible lesion to encompass any locations of positive nodes comprising cancerous tissue to generate a minimum treatment volume (MTV) 3D model; at least partially expanding the MTV 3D model to cover any potentially cancerous tissue; and at least partially contracting the MTV 3D model to exclude any 206030-0074-P1-US.604931 locations of negative nodes to generate an Optimized Margin 3D model.

In one embodiment, the MTV 3D model margin is at least partially expanded to encompass the location of neutral nodes. In one embodiment, the MTV 3D model margin is isotropically expanded by 1 cm in all directions. In one embodiment, the MTV 3D model is at least partially expanded to encompass regions that appear to be cancer harboring based on medical image data. In one embodiment, the MTV 3D model is at least partially expanded to encompass cancer-containing regions based on statistical analysis of a population of previous biopsies, a population of previously treated patients, or both.

In another aspect, the present invention relates to a system for focal laser therapy of soft tissue, comprising a laser; at least one thermal sensor; a needle guide; an ultrasound probe; a 3D scanning and location tracking assembly; and a computer platform.

In one embodiment, the system further comprises at least one optical sensor. In one embodiment, the system further comprises at least one multi-modal sensor having at least one thermal sensing element and at least one optical sensing element.

In one embodiment, the laser comprises a laser fiber, a coolant, a dual lumen catheter, a cooling pump, a flow sensor, and a flow controller. In one embodiment, the laser fiber is capable of emitting between 5 and 50 W of light. In one embodiment, the coolant is an inert solution of water or saline. In one embodiment, the coolant is room temperature. In one embodiment, the coolant is room temperature or below room temperature.

In another aspect, the present invention relates to a multi-channel needle guide device, comprising an elongate body; a first channel having a first channel centerline; an auxiliary channel having an auxiliary channel centerline; and a plurality of attachment clips.

In one embodiment, the device further comprises a locking member selected from the group consisting of: a screw, a clamp, a bolt, and a pin. In one embodiment, the plurality of attachment clips comprises tabs, hooks, or slots to secure the multi-channel needle guide device to the body of an ultrasound probe.

In one embodiment, the first channel has a lumen sized suitably for a biopsy needle, catheter, laser fiber, or trocar to pass therethrough. In one embodiment, the auxiliary channel has a lumen sized suitably for a thermal sensor, an optical sensor, or a multi-modal sensor to pass therethrough. In some embodiments, the first channel centerline and the auxiliary channel centerline are spaced between 1 and 20 mm apart. In one embodiment, the device further comprises at least one additional auxiliary channel.

In another aspect, the present invention relates to a method of focal laser therapy of soft tissue. The method comprises the steps of: capturing a real-time 3D ultrasound model of a patient's region of interest to be treated; overlaying at least one cancer margin 3D model over the real-time 3D ultrasound model; generating at least one expected damage model, wherein the at least one expected damage model at least partially overlaps the at least one cancer margin 3D model; calculating at least one laser fiber location in the patient's region of interest and at least one ablation setting to fit the at least one expected damage model, wherein the at least one ablation setting comprises a laser power output, a laser exposure duration, a laser exposure rate, and a coolant flow rate; calculating at least one sensor location in the patient's region of interest; inserting a laser fiber into the at least one laser fiber location and at least one sensor into the at least one sensor location; executing the at least one ablation setting; and monitoring treatment progression by modeling the extent of tissue damage.

In one embodiment, the at least one cancer margin 3D model comprises a MRI-visible lesion 3D model, a MTV 3D model, an Optimized Margin 3D model, and biopsy core location. In one embodiment, the expected damage model comprises three nested ellipsoids, the smallest ellipsoid representing minimum expected damage (minED), the medium ellipsoid representing average expected damage (aveED), and the largest ellipsoid representing maximum expected damage (maxED). In one embodiment, the minED of the expected damage model encapsulates the entirety of the MTV 3D model.

In one embodiment, the at least one sensor comprises at least one thermal sensor, at least one optical sensor, at least one multi-modal sensor, or any combination thereof. In one embodiment, the ablation settings are limited from generating a temperature in excess of 95° C. In one embodiment, the extent of tissue damage is modelled by measuring the temperature of tissue adjacent to the region of interest being treated. In one embodiment, the extent of tissue damage is modelled by measuring the rate of tissue cooling immediately after executing the at least one ablation setting. In one embodiment, the extent of tissue damage is modeled by ultrasound measurements of tissue temperature change, mechanical property change, or vascularity change. In one embodiment, the extent of tissue damage is modeled by measuring the amount of light scatter in the region of interest being treated. In one embodiment, the extent of tissue damage is modeled by quantifying the level of thermally induced alterations in tissue optical properties.

In another aspect, the present invention relates to a multi-modal sensor probe comprising: an elongate central thermal sensor; at least two optical fibers positioned adjacent and parallel to the central thermal sensor; a prism positioned at one end of each optical fiber; and a housing encasing the central thermal sensor, the at least two optical fibers, and the prisms.

In another aspect, the present invention relates to a multi-modal sensor probe comprising: at least one optical fiber, each optical fiber adjacent and parallel to each other; a temperature-sensitive material, positioned at one end of each optical fiber; and a housing encasing the at least one optical fiber and the temperature-sensitive material, wherein the temperature-sensitive material is phosphor.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 is a flowchart depicting an exemplary method of cancer margin determination for focal laser ablation of soft tissue.

FIG. 2 is a diagram of an exemplary system for focal laser ablation of soft tissue.

FIG. 3 is a diagram of an exemplary multimodal sensor probe tip.

FIG. 4 is a diagram of another exemplary multimodal sensor probe tip. FIG. 5 is a diagram depicting several views of an exemplary multi-channel needle guide having two channels.

FIG. 6 is a diagram depicting an exemplary multi-channel needle guide having two channels from an anterior perspective and a conceptual representation of multi-channel orientation.

FIG. 7 is a diagram depicting the insertion of a biopsy needle into the first channel of an exemplary multi-channel needle guide having two channels.

FIG. 8 depicts top and bottom views of an exemplary multi-channel needle guide having two channels with a dual lumen catheter inserted into the first channel and a catheter inserted into the auxiliary channel.

FIG. 9 depicts the use of an exemplary multi-channel needle guide having two channels and an exemplary multi-channel needle guide having three channels to treat a prostate, each using at least one multiple-temperature or other sensing element in the auxiliary channel(s).

FIG. 10 is a flowchart depicting an exemplary method of focal laser therapy of soft tissue.

FIG. 11 depicts a diagram showing exemplary intra-prostatic placement of a laser fiber and three thermal probes. The laser fiber is inserted trans-rectally, and the thermal probes are inserted transperineally. The thermal probes are used for independent measurement of temperatures at the margin of the treatment zone (probes 1 and 2) and near the rectal wall (probe 3), as seen on axial inset. During treatment, intra-prostatic temperature is continuously monitored and recorded by MR-thermometry (every 6 seconds) and by the thermal probes (real-time) via a multi-channel reorder. Position of the fiber and thermistors was periodically re-confirmed by MR scanning during each treatment.

FIG. 12 is a table listing the baseline characteristics of the 10 men treated. An 11th patient was excluded because laser fiber could not be positioned.

FIG. 13 is a table listing the adverse events of each patient graded by the Common Terminology Criteria for Adverse Events (CTCAE) version 4.03. All patients were discharged home within 1-2 hours.

FIG. 14 is a table listing the Gleason score and maximum cancer core length for each patient before and after focal laser ablation (FLA).

FIG. 15 depicts an exemplary room setup for FLA in an outpatient clinic procedure room.

FIG. 16 depicts an exemplary Artemis fusion device arm providing a stable platform for securing and repositioning a laser fiber (red) and thermal probe (white) during treatment

FIG. 17 depicts a diagram showing the relationship between the laser fiber (yellow) and thermal probes (blue) in the prostate during FLA. Laser fiber is inserted transrectally and thermal probes are inserted transrectally and transperineally. The tumor is shaded in green. Thermal probes provide continuous monitoring of intra-prostatic temperature throughout the procedure. Appropriate positioning of the laser fiber within the prostate is verified during the procedure with real-time ultrasound.

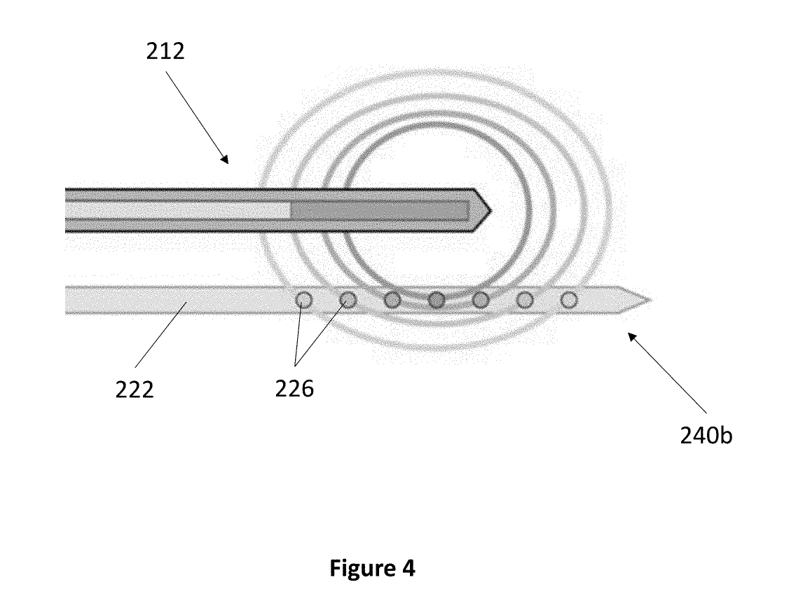

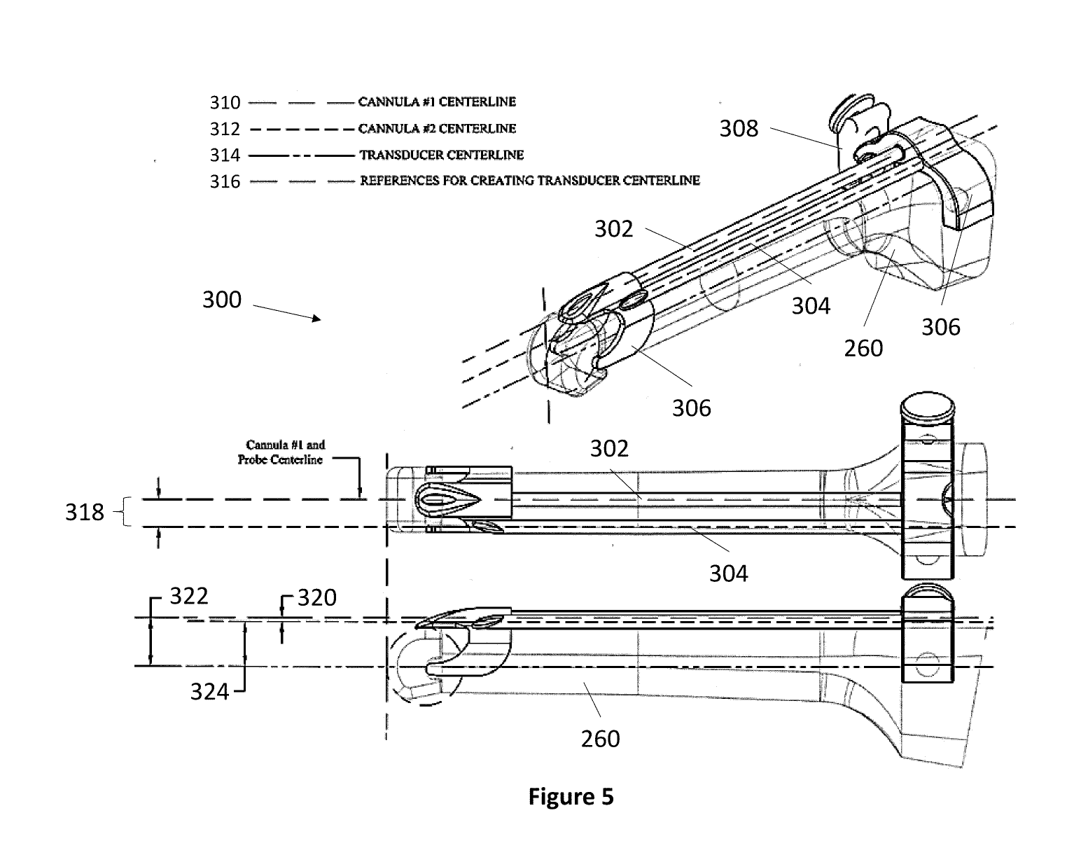

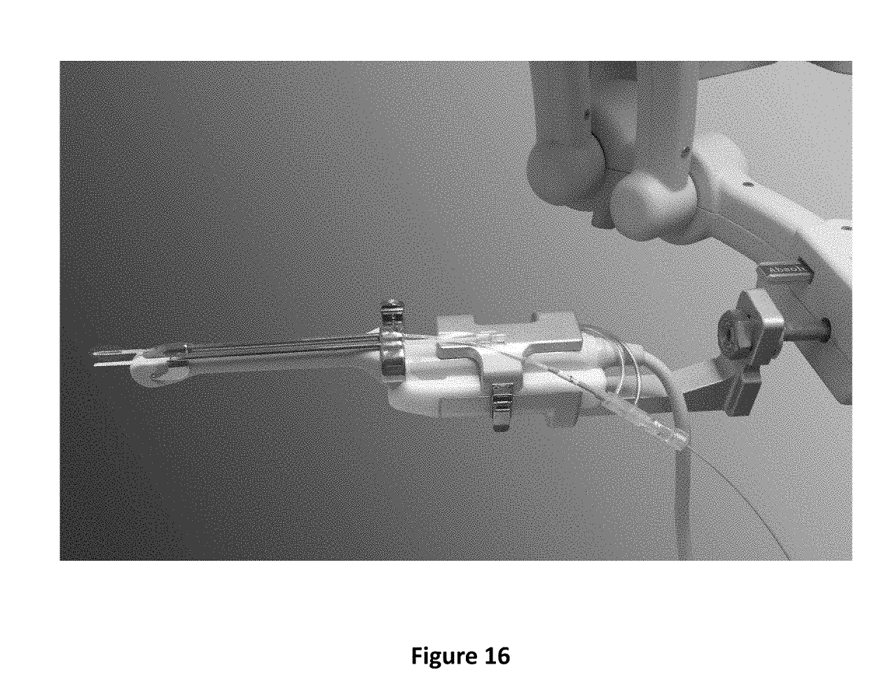

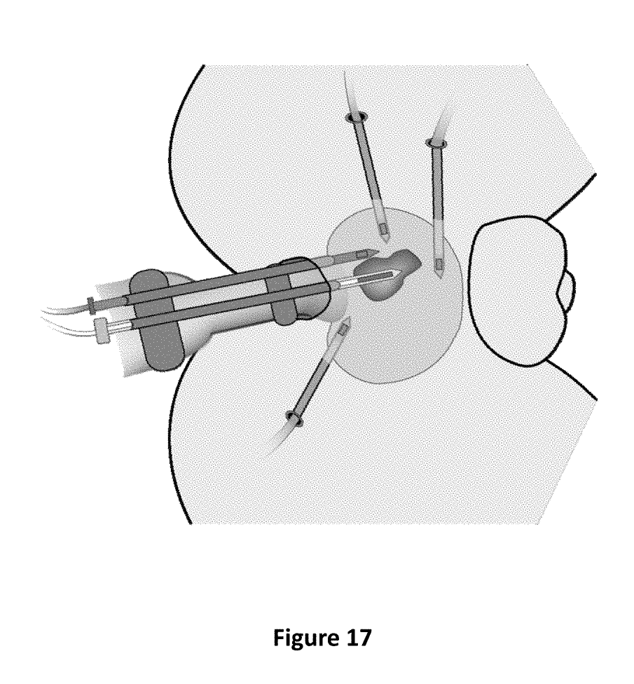

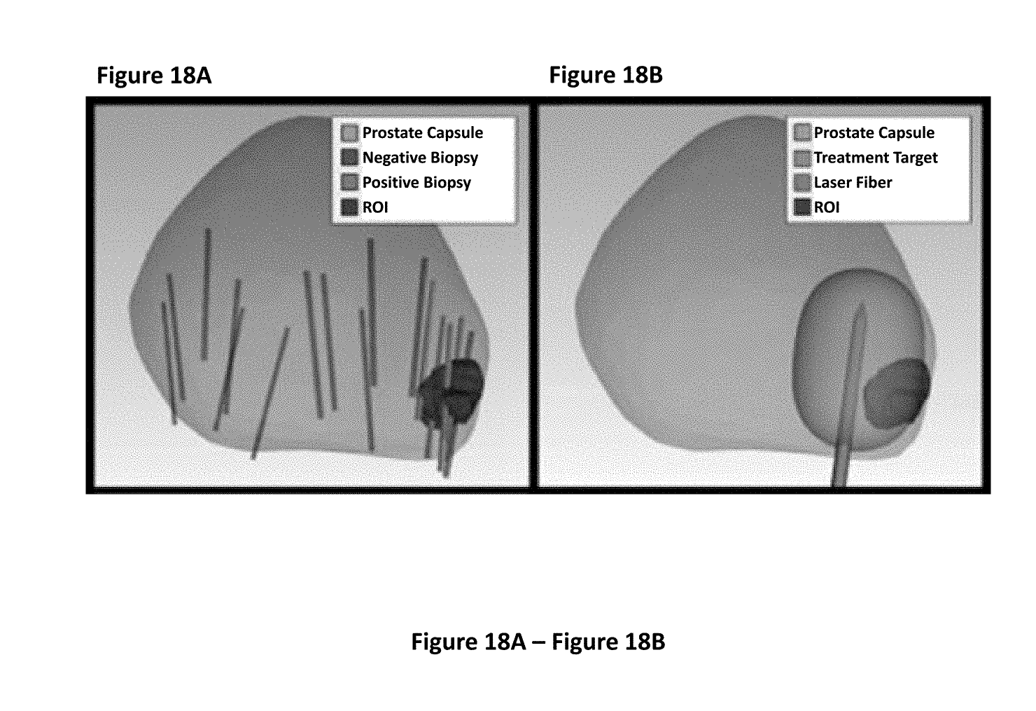

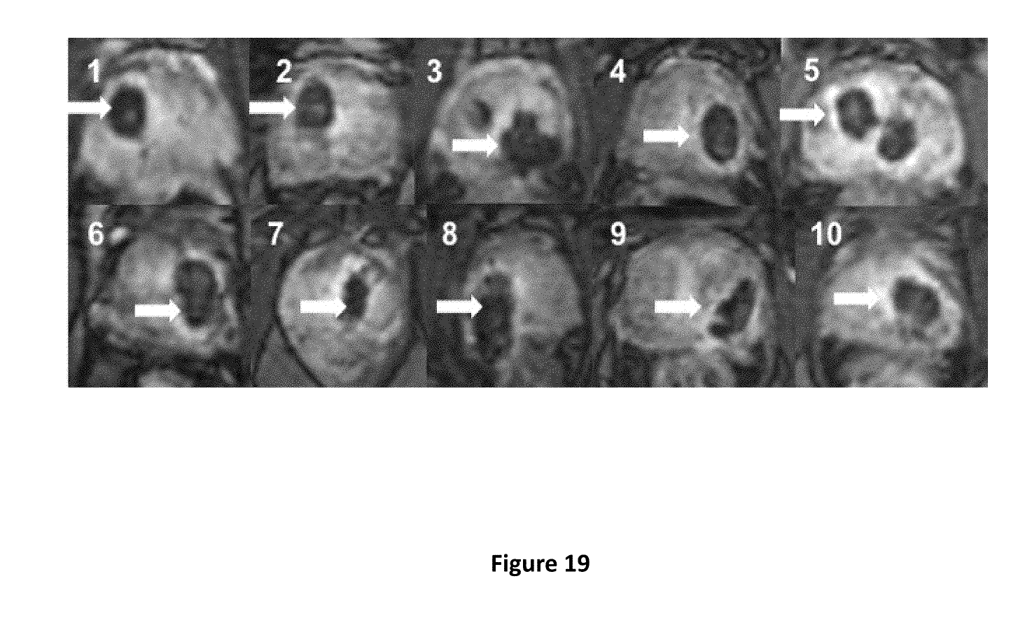

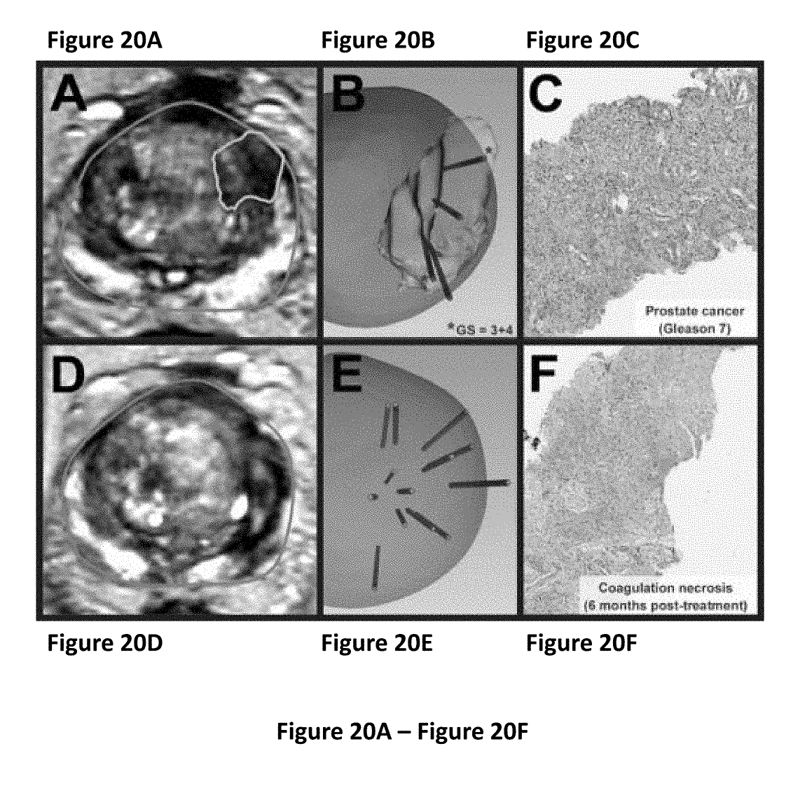

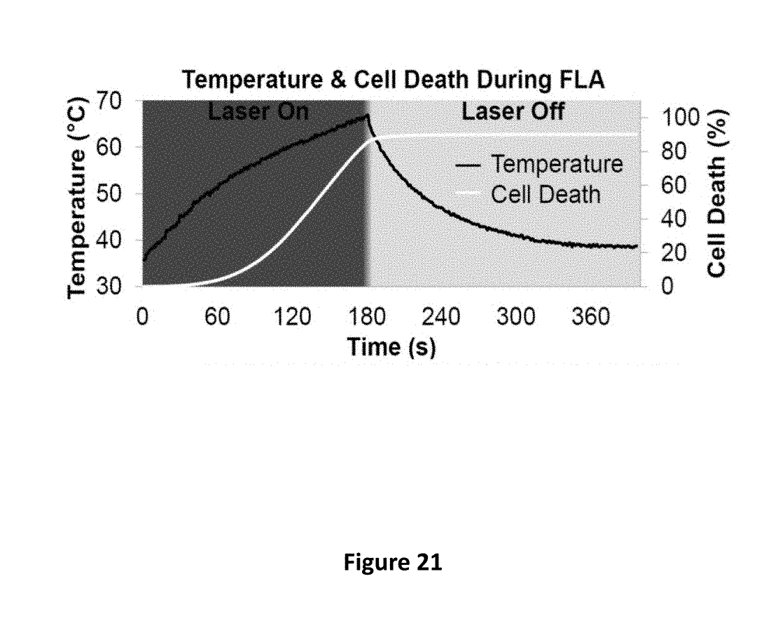

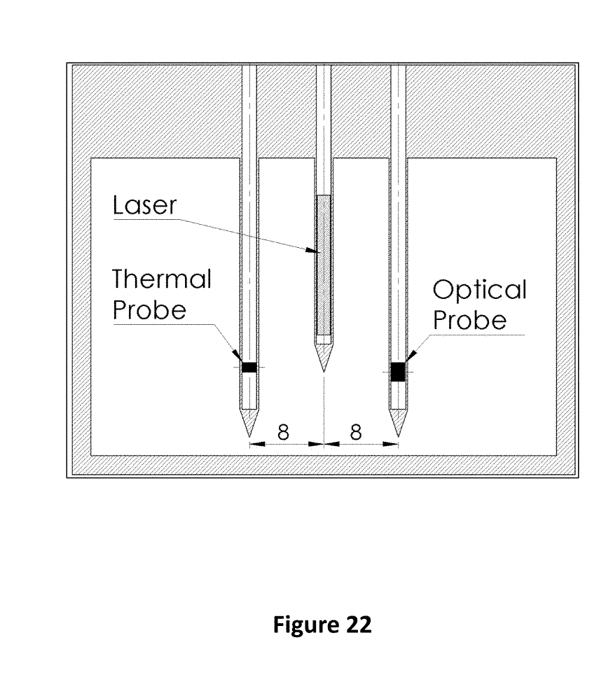

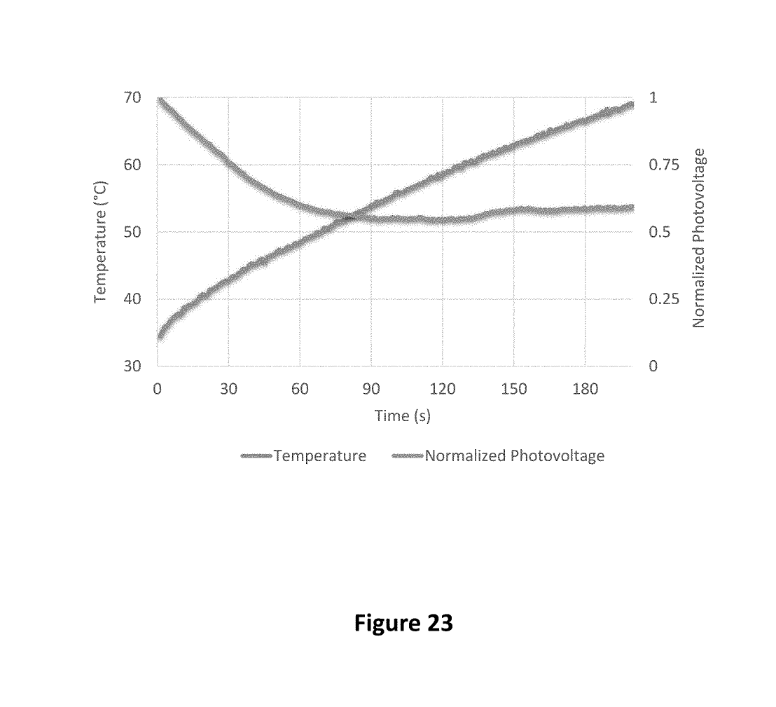

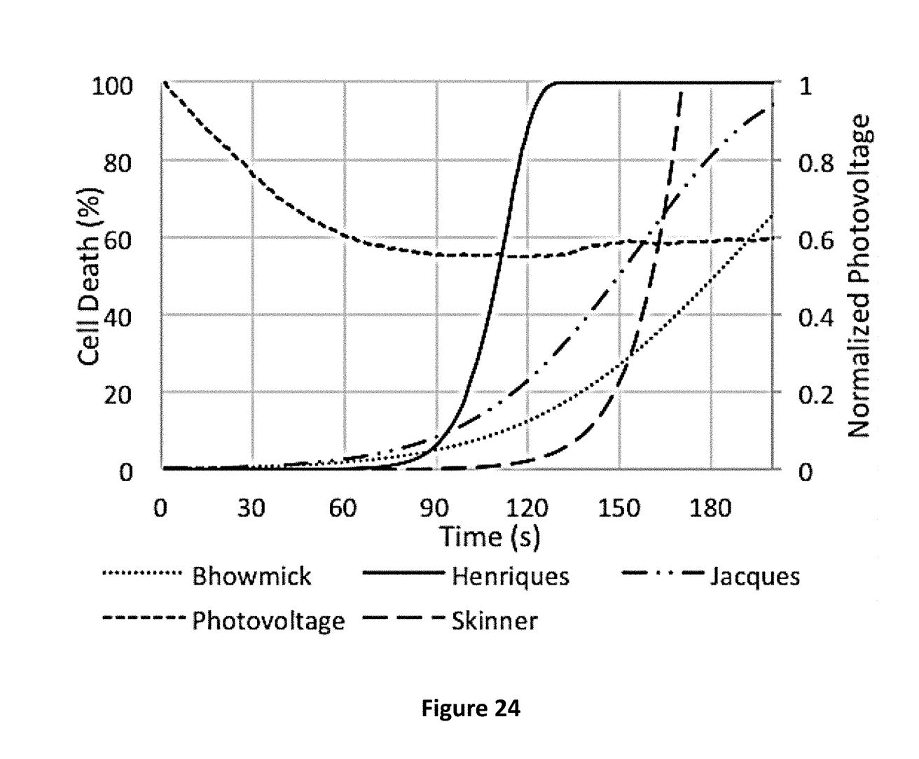

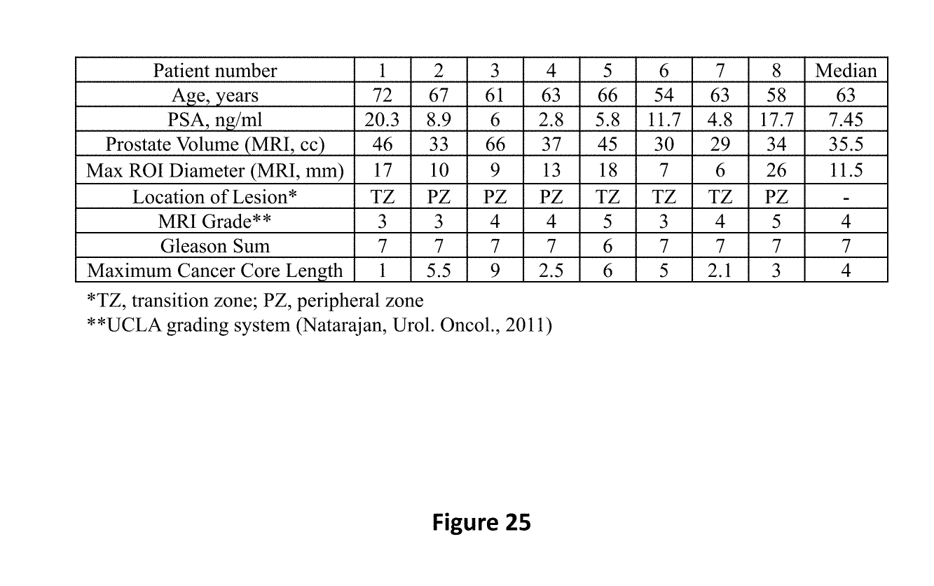

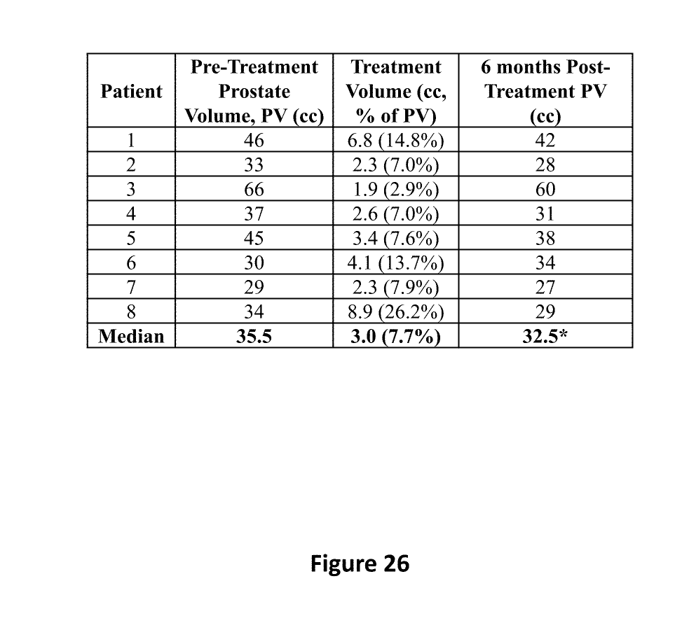

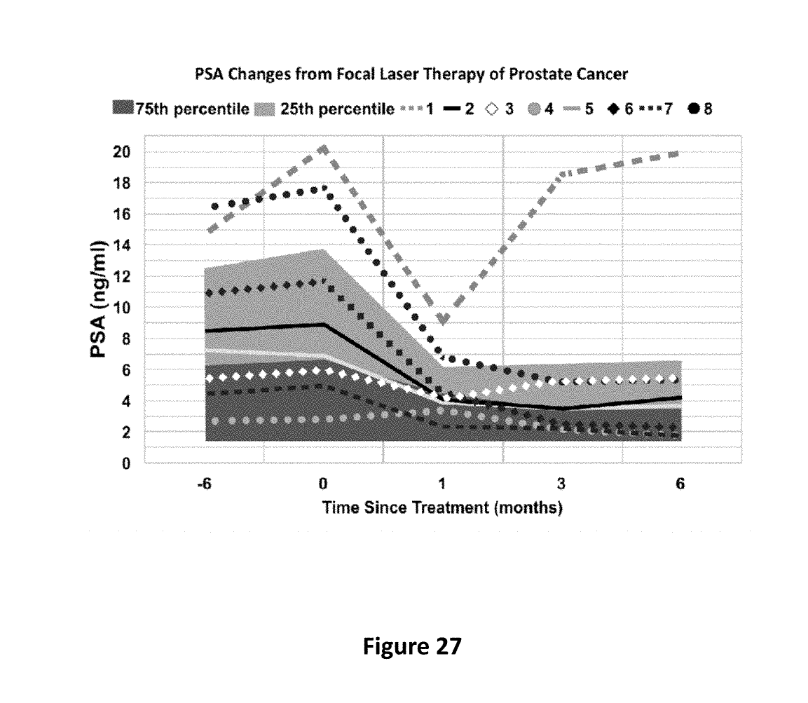

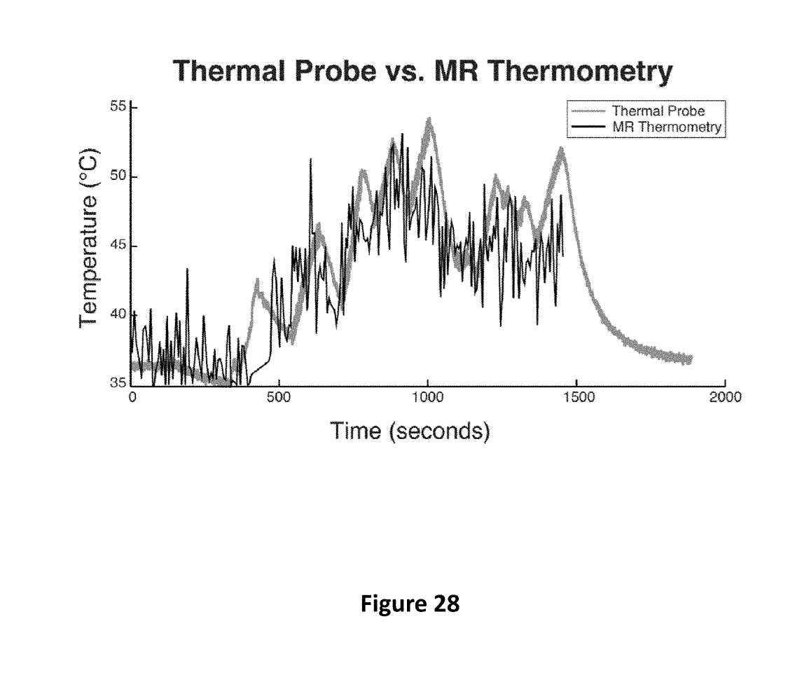

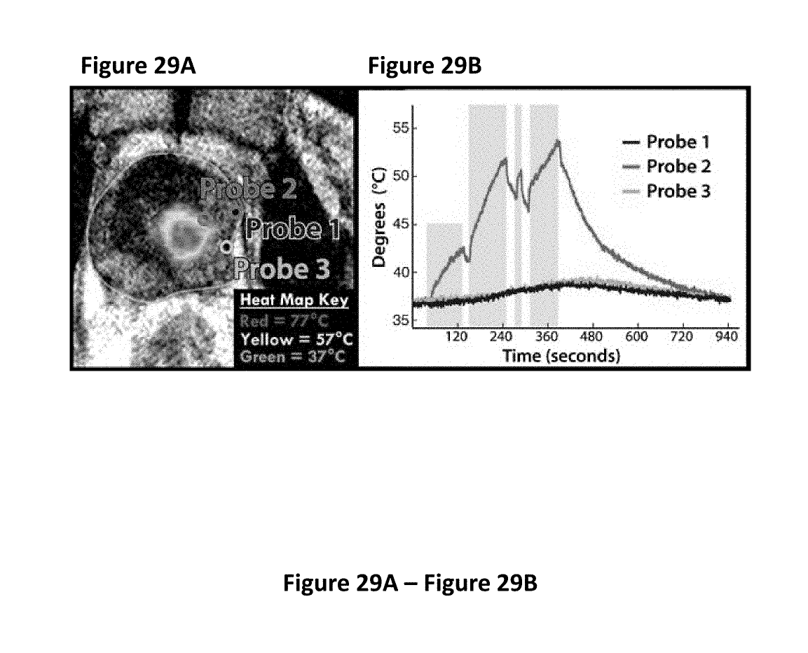

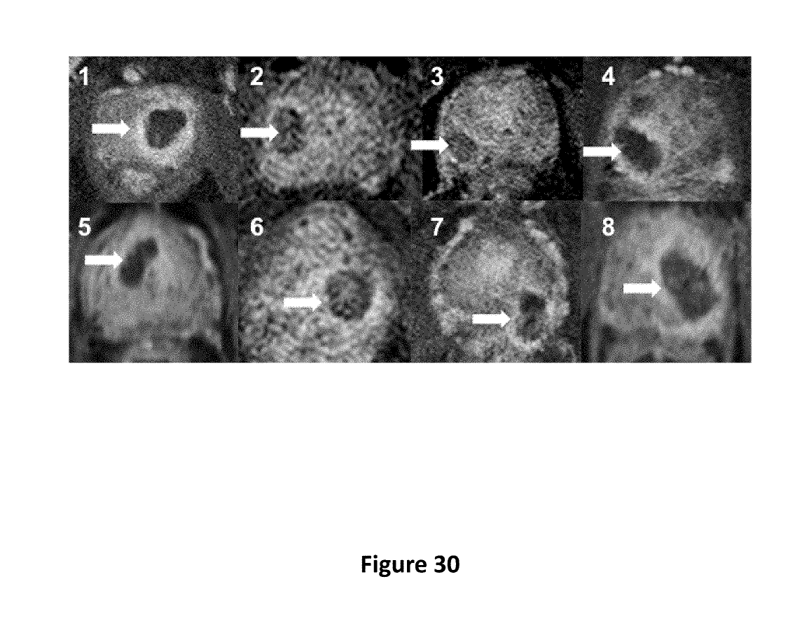

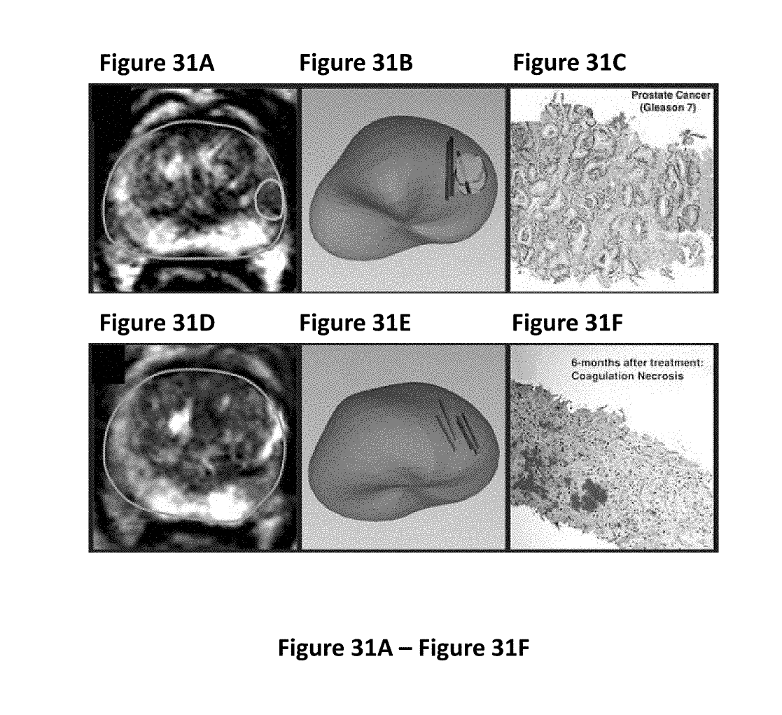

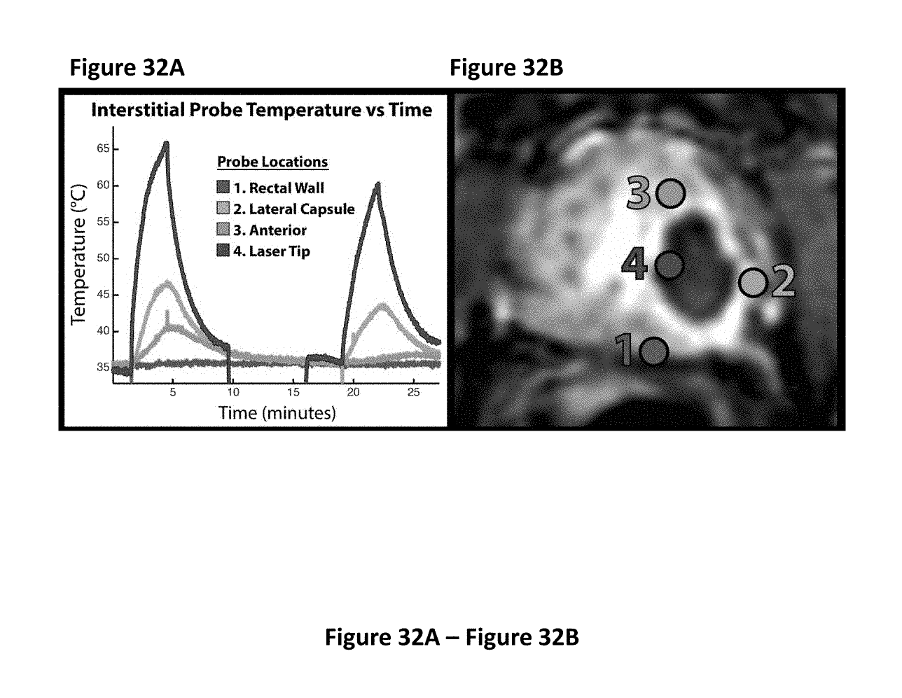

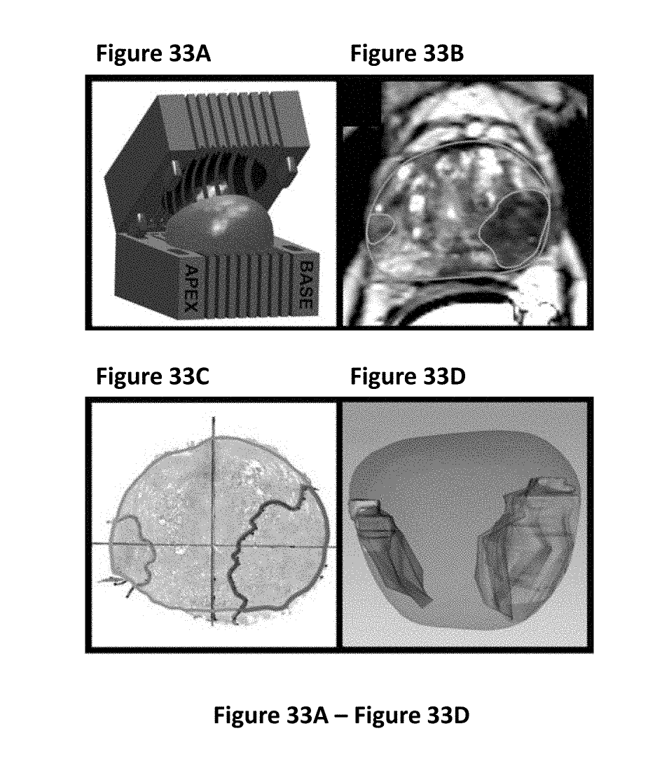

FIG. 16A and FIG. 16B depict the determination of a region of interest. (FIG. 4A) 3D prostate model of fusion biopsy showing regions of interest with positive and negative cores. (FIG. 4B) Patient-specific 3D prostate model used to estimate treatment size of FLA treatment.

FIG. 19 depicts a series of dynamic contrast-enhanced (DCI) MRI showing localized hypo-perfusion of the ablation zone in all 10 patients in the original site of biopsy-confirmed tumor.

FIG. 20A through FIG. 20F depict the imaging and histologic findings of patient 6. Before treatment MRI showed a grade 4 ROI (FIG. 20A) which on MRI/US fusion biopsy (FIG. 20B) revealed Gleason 3+4=7 CaP (FIG. 20C). Six months after FLA, ROI is no longer visible on MRI (FIG. 20D). MRI/US fusion biopsy of prostate revealed no cancer (FIG. 20E), only coagulation necrosis (FIG. 20F). Among the last 6 men treated, a similar result was found in 3.

FIG. 21 depicts the change in temperature and percent cell death during an in vivo laser interstitial thermal therapy (LITT). Cell death is estimated using the Arrhenius integral approach.

FIG. 22 is a diagram depicting the experimental setup for testing an optical monitoring system.

FIG. 23 depicts the change in temperature and photovoltage during LITT.

FIG. 24 compares the change in photovoltage against several damage estimations during LITT.

FIG. 25 is a table depicting the baseline characteristics of men enrolled in the focal laser ablation (FLA) trial. At baseline, at least 10 systematic biopsy cores were obtained to exclude multi-focality, and at least 2 cores were obtained from the MR-visible region of interest, i.e., the lesion to be treated. *TZ, transition zone; PZ, peripheral zone. **UCLA grading system (Natarajan et al., Urol Oncol, 2011, 29(3):334-342).

FIG. 26 is a table depicting the MRI changes within 4 hours and within 6 months of FLA treatment. Treatment volume, as determined by the non-perfused region seen on immediate post-treatment multi-parametric MRI (mpMRI), was a median of 3 cc (7.7% of the prostate volume). *Prostate volumes on six month post-FLA MRI significantly decreased compared to pre-treatment volumes (p=0.03, Wilcoxon sign-rank test).

FIG. 27 is a graph depicting the amount of prostate-specific antigen over time for all 8 men treated with focal laser therapy, showing values prior to screening (˜6 months), prior to FLA treatment (0 months), and at post-treatment follow-up (1, 3, 6 months). PSA significantly dropped from a median value of 8 ng/mL to 3.3 ng/mL six months after FLA (p=0.0078). A significant drop in PSA density and increase in free PSA was also observed.

FIG. 28 is a graph depicting MRI thermometry (MRT) (black) versus thermal probe recordings (grey) of focal therapy patient #6, at a point 13 mm from the laser tip. Temperatures reported by MRT were unreliable and noisy in every case, largely due to motion artifacts. MRI scanning ceased at 1500 seconds, while thermal probes continued to report data. MRT scan parameters: (repetition time=24 ms; echo time=10 ms; field of view=220×220 mm; flip angle=25 degrees; slice thickness=4 mm; resolution=0.86×0.86 mm).

FIG. 29A and FIG. 29B depict temperature changes in a prostate during FLA. FIG. 29A depicts an MRI of focal therapy patient #8, overlaid with filtered thermometry map, showing thermal probe positions. Heat from the laser fiber is confined, i.e., limited to a contained area around the laser tip. FIG. 29B depicts a chart of temperature changes recorded by thermal probes. Temperature probe 1 (16.6 mm from the laser tip) and probe 3 (14.4 mm from the laser tip) show little change in temperature, while probe 2 (8.2 mm from the laser tip) records considerable heating during the activation periods (vertical bars).

FIG. 30 depicts images of dynamic contrast enhancement MRI in all 8 patients within 2 hours of focal laser ablation. A well-defined under-perfused region (white arrows) indicates that treatment was confined to the target region, away from critical structures.

FIG. 31A through FIG. 31F depict the prostate of focal therapy patient #6 before and after FLA treatment. Before treatment, MRI showed a Grade 4 region of interest (FIG. 31A), which upon targeted biopsy (FIG. 31B), revealed Gleason 3+4=7 prostate cancer (FIG. 31C). Six months after FLA, the original region of interest is no longer visible (FIG. 31D). Targeted prostate biopsies from the treatment zone (FIG. 31E) showed no cancer, only areas of coagulation necrosis and old hemorrhage (FIG. 31F). Screening and follow-up systematic biopsies and cores from the margin of the treatment zone (not pictured) also were negative for prostate cancer.

FIG. 32A is a graph depicting interstitial probe temperatures during FLA treatment. Probes farther from the non-perfused region experience lower temperatures, assuring minimal damage to surrounding tissue.

FIG. 32B is a post-treatment dynamic contrast-enhancement image showing the treated region as non-perfused.

FIG. 33A through FIG. 33D depict the process of precision sectioning a prostate. A 3D-printed patient-specific mold (FIG. 33A) was used to correlate mpMRI (FIG. 33B) with whole-mount pathology (FIG. 33C) and to perform 3D co-registration (FIG. 33D) and contribute to a database for determination of treatment margins.

FIG. 34 is a graph depicting Gleason scores of tumors stratified by MRI suspicion level (UCLA Grade 3-5), demonstrating increasing cancer severity as MR suspicion rises.

FIG. 35 is a table depicting the accuracy of pre-op mpMRT for detection of prostate cancer and clinically significant prostate cancer in 65 men. Patient-specific molds were used to correlate whole mount slides with MM.

FIG. 36A though FIG. 36C depict the co-registration of tumor pathology (FIG. 36A) with MRI (FIG. 36B). In FIG. 36C, the irregular contour and maximum extent of the tumor beyond a matched ROI is shown. Significant MRI underestimation of both tumor volume and longest tumor axis is apparent.

FIG. 37 is a table depicting the spatial parameters of prostates and matched tumors as determined by MRI vs. whole mount pathology sections (N=71 tumors, 65 prostates). MRI significantly underestimated tumor volume and longest axis (matched pair t-test, p<0.01).

### DETAILED DESCRIPTION

The present invention provides methods of determining cancer margins indicating the location and breadth of treatment necessary for the elimination of cancerous tissue during focal laser therapy. The present invention also provides systems and devices for focal laser therapy, and methods for using the same. The present invention does not rely on MRI thermometry, improving accuracy of treatment while also reducing treatment time and cost.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Method of Cancer Margin Determination for Focal Laser Therapy of Soft Tissue

In one aspect, the present invention provides a method of cancer margin determination. The method combines radiology data and pathology data to generate 3D models representing the location and breadth of cancerous tissue for focal laser therapy.

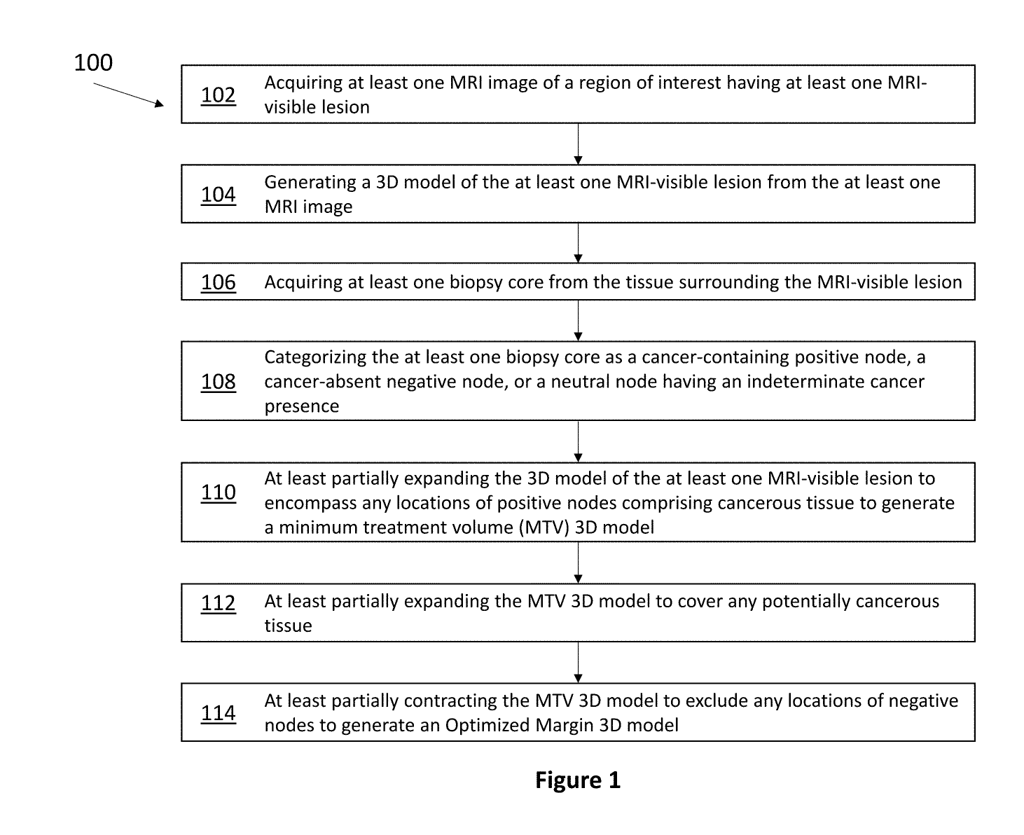

Referring now to FIG. 1, an exemplary method 100 of cancer margin determination is presented. Method 100 begins with step 102, wherein at least one MM image of a region of interest having at least one MRI-visible lesion is acquired. In step 104, a 3D model of the at least one MM-visible lesion is generated from the at least one MRI image. In step 106, at least one biopsy core is acquired from the tissue surrounding the MRI-visible lesion. In step 108, the at least one biopsy core is categorized as a cancer-containing positive node, a cancer-absent negative node, or a neutral node having an indeterminate cancer presence. In step 110, the 3D model of the at least one MRI-visible lesion is at least partially expanded encompass the locations of positive nodes comprising cancerous tissue to generate a minimum treatment volume (MTV) 3D model. In step 112, the MTV 3D model margin is at least partially expanded. In some embodiments, the MTV 3D model margin is isotropically expanded. In other embodiments, the MTV 3D model is expanded to include at least one neutral node, or to include any regions or structures visible from MRI or US imaging that appear suspicious. In step 114, the MTV 3D model is at least partially contracted to exclude the locations of negative nodes to generate an Optimized Margin 3D model.

A region of interest refers to a region of soft tissue comprising cancerous tissue. The method of acquiring at least one MRI image of a region of interest can be any suitable MRI method commonly used in the art. In some embodiments, the MRI method comprises multi-parametric MRI. The method of generating 3D models of the at least one MRI-visible lesion may be performed using any suitable software capable of collating a plurality of MRI images into a three-dimensional representation. The 3D model allows an operator to spatially visualize the size, shape, and location of the at least one MRI-visible lesion. The 3D model also allows an operator to plan the acquisition of the at least one biopsy core from the tissue surrounding the MRI-visible lesion, such that the biopsy cores avoid sensitive anatomical structures while capturing a representative sampling of the local tissue.

The method of acquiring the at least one biopsy core can be any suitable method known in the art, including ultrasound (US) guided methods using biopsy core needles having a needle gauge between 12 and 20. Typical biopsy cores comprise a diameter and a length, wherein the spatial location of cancerous tissue may be determined both by the source of the biopsy core in the origin tissue and by the position of the cancerous tissue along the length of a biopsy core. Labeling the at least one biopsy core as a cancer-containing positive node, a cancer-absent negative node, or a neutral node having an indeterminate cancer presence enables an operator to discern the actual boundaries of the cancer in the region of interest that is not visible in the MRI images.

The biopsy core samples may reveal cancer-containing tissue in locations outside of the 3D models of the at least one MRI-visible lesion. The 3D models may be deformed by an operator to address the absence of the positive nodes. For example, the operator may introduce bulges into the 3D model to envelope positive nodes, wherein a 3D model enveloping all positive nodes represents an MTV 3D model.

The expansion of the MTV 3D model is non-rigid and can be freely deformed by an operator to better fit the actual boundaries of cancerous tissue in the region of interest. To capture cancerous tissue in the region of interest that is not clearly visible with MRI, the MTV 3D model may be further expanded. In one embodiment, the MTV 3D model is expanded isotropically in all directions. The amount of expansion may vary depending on many factors including lesion location, tissue type, lesion type, and the like. For instance, for a lesion that is in close proximity to a sensitive anatomical structure, the 3D model expansion may be in all directions except in the direction of the sensitive anatomical structure. In another example, a lesion comprising a high level of vasculature may warrant a larger amount of expansion than a lesion comprising benign cancerous tissue with a low level of vasculature. In some embodiments, the MTV 3D model is isotropically expanded by 1 cm in all directions.

In some embodiments, the MTV 3D model is expanded based on the likelihood of any tissue region or structure to harbor cancer. For example, the 3D model may be expanded anisotropically based on statistical analysis of cancer locations in a population of previous biopsies, a population of treated patients, or both.

The MTV 3D model may overlap at least one negative biopsy node. The 3D model may be deformed by an operator to address the presence of negative nodes. For example, the operator may introduce dimples or depressions into the 3D model to exclude negative nodes, wherein the resulting 3D model represents an Optimized Margin 3D model.

### System for Focal Laser Therapy of Soft Tissue

In another aspect, the present invention provides a system for focal laser therapy of soft tissue. The system does not require the use of MRI thermometry while still enabling real-time monitoring of temperature and treatment progress, reducing the time and resources required to administer focal laser therapy of soft tissue.

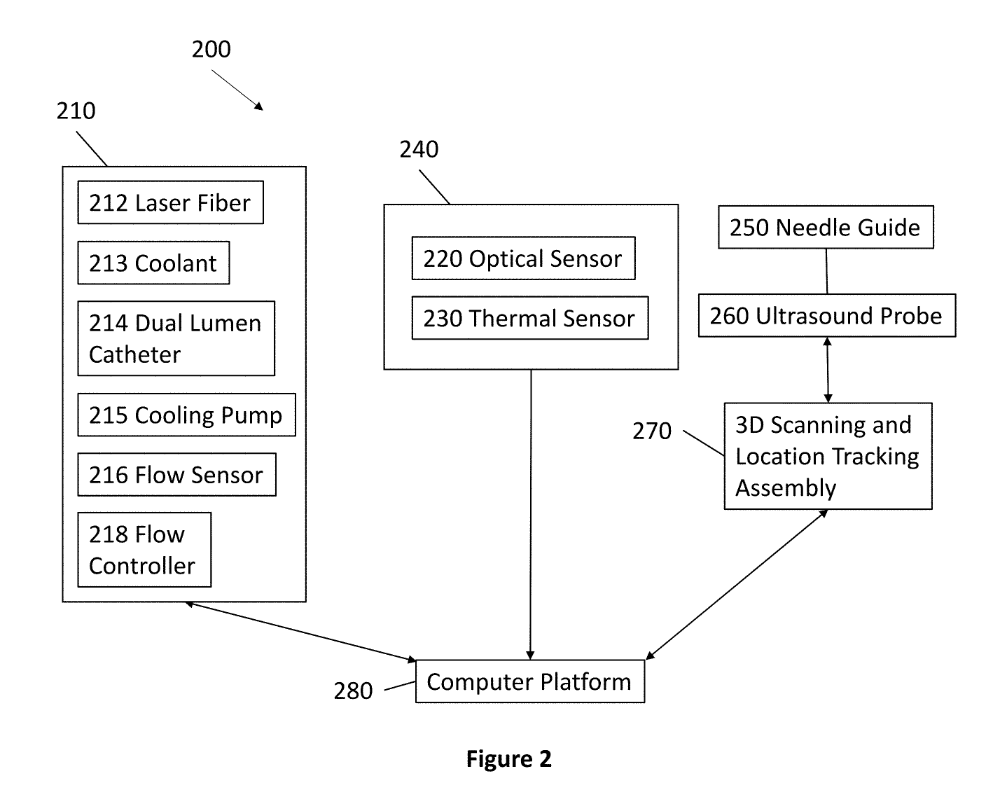

Referring now to FIG. 2, a diagram of an exemplary system 200 for focal laser therapy of soft tissue is depicted. System 200 comprises laser 210, at least one optical sensor 220, at least one thermal sensor 230, needle guide 250, ultrasound probe 260, 3D scanning and location tracking assembly 270, and computer platform 280.

Laser 210 comprises laser fiber 212, coolant 213, dual lumen catheter 214, cooling pump 215, flow sensor 216, and flow controller 218. Laser fiber 212 can be any suitable laser fiber capable of guiding laser light to the target and emitting it with sufficient power to cause coagulative necrosis. In some embodiments, laser fiber 212 comprises a diffusing or reflecting element at its tip for focal direction of light. In some embodiments, a suitable laser fiber is capable of transporting and emitting between 5 and 50 W of light. Higher laser energy outputs are supported by active cooling, wherein coolant 213 is circulated adjacent to the laser fiber by cooling pump 215 and controlled by flow sensor 216, and flow controller 218. In one embodiment, active cooling is achieved by inserting laser fiber 212 into a first lumen of dual lumen catheter 214 and circulating coolant 213 through a second lumen of dual lumen catheter 214. Coolant 213 can be any suitable coolant used in the art, such as an inert solution of water or saline. In one embodiment, coolant 213 is room temperature. In another embodiment, coolant is below room temperature. Flow controller 218 modulates the rate of coolant circulation, while flow sensor 216 actively tracks the rate of coolant circulation and alerts the operator in the event of a problem, such as a restricted flow of coolant. In some embodiments, lasers commonly used in the art may be incorporated into system 200, such as the Visualase laser thermal ablation system.

The at least one optical sensor 220 and the at least one thermal sensor 230 provide means for real-time monitoring of the performance of laser 210 and treatment progress. The at least one optical sensor 220 can be any suitable sensor that can measure laser fluence or laser radiance in vivo. For example, an optical fiber may be used to deliver light from the region of interest to a photodiode. Likewise, the at least one thermal sensor 230 can be any suitable sensor that can measure temperature in vivo, such as a thermistor or a fluoroptic sensor. In some embodiments, system 200 comprises at least one multimodal sensor 240, which combines at least one optical sensing element and at least one thermal sensing element into a single device.

Needle guide 250 comprises at least one linear channel to guide the direction of instrument insertion. For example, the at least one channel of needle guide 250 may accept instruments such as laser fiber 212, optical sensor 220, thermal sensor 230, multi-modal sensor 240, biopsy needles, or trocars for accurate placement within a region of tissue. In some embodiments, needle guide 250 is a multi-channel needle guide, as described elsewhere herein. In some embodiments, needle guide 250 can be at least partially attached to ultrasound probe 260. Attaching needle guide 250 to ultrasound probe 260 allows an operator to manipulate both devices at once.

3D scanning and location tracking assembly 270 converts ultrasound images sent from ultrasound probe 260 into 3D models. The 3D models enable an operator to visualize a region of tissue that is being treated, as well as the spatial orientation of any devices that are inserted into the region of tissue. 3D scanning and location tracking assembly 270 further comprises means for controlling the spatial orientation of any devices that are inserted into the region of tissue, such as ultrasound probe 260 and needle guide 250. An exemplary 3D scanning and location tracking assembly 250 includes the Artemis MRI/Ultrasound Fusion Device (Eigen, Grass Valley, Calif.).

As contemplated herein, computer platform 280 may comprise any computing device as would be understood by those skilled in the art, including desktop or mobile devices, laptops, desktops, tablets, smartphones or other wireless digital/cellular phones, televisions or other thin client devices as would be understood by those skilled in the art.

Computer platform 280 is fully capable of sending commands to the components of system 200 and interpreting received signals as described herein throughout. In certain embodiments, portions of the system may be computer operated, or in other embodiments, the entire system may be computer operated. The computer platform can be configured to control parameters such as coolant flow rate, laser power output, and ultrasound frequency, intensity, amplitude, period, wavelength, and the like. The computer platform can also be configured to control the actuation of devices with 3D scanning and location tracking assembly 270, including parameters such as angulation and partial locking. The computer platform can be configured to record received signals, and subsequently interpret the received signals in real-time. For example, the computer platform may be configured to interpret the received signals as images and subsequently transmit the images to a digital display. The computer platform may further perform automated calculations based on the received signals to output data such as density, distance, temperature, composition, imaging, treated volume, and the like. The computer platform may further provide a means to communicate the received signals and data outputs, such as by projecting one or more static and moving images on a screen, emitting one or more auditory signals, presenting one or more digital readouts, providing one or more light indicators, providing one or more tactile responses (such as vibrations), and the like. In some embodiments, the computer platform communicates received signals and data outputs in real-time, such that an operator may adjust the use of the device in response to the real-time communication. For example, in response to a signal from flow sensor 216 indicating restricted coolant flow, the computer platform may decrease the output of laser fiber 212 or direct 3D scanning and location tracking assembly 270 to extract laser fiber 212 from a patient to prevent injury.

The computer platform may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

The software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert an operator of the particular results. Further embodiments of such mechanisms are described elsewhere herein or may be standard systems understood by those skilled in the art.

### Multimodal Sensor Probe

In another aspect, the present invention provides a multimodal sensor probe. The multimodal sensor probe provides enhanced monitoring of tissue ablation during the performance of the methods of the present invention.

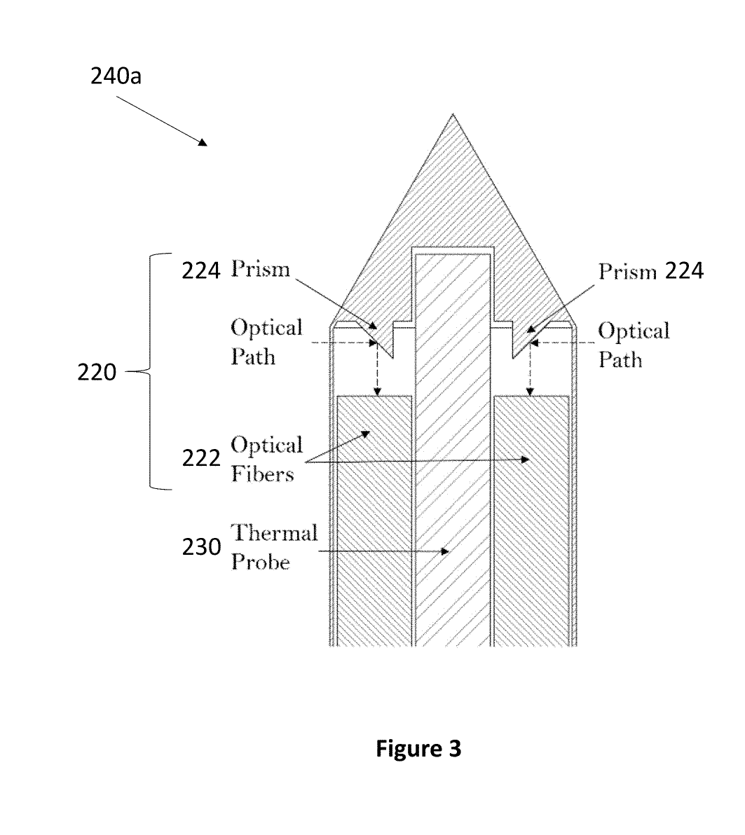

Referring now to FIG. 3, an exemplary multimodal sensor 240a is depicted. Multimodal sensor 240a comprises an elongate casing having at least one lumen for holding one or more sensors. For example, as described elsewhere herein, in one embodiment multimodal sensor 240a comprises at least one optical sensor 220 and at least one thermal sensor 230 arranged in parallel within the lumen of multimodal sensor 240a. In FIG. 3, multimodal sensor 240a comprises two optical sensors 220, each having an optical fiber 222 and a prism 224. Optical fibers 222 are placed opposite to one another, such that the prisms 224 direct light having radiance angles of 0° (facing a laser diffuser) and 180° (facing away from a laser diffuser). Thermal sensor 230 comprise a fluoroptic thermal probe positioned between optical fibers 222.

Referring now to FIG. 4, an exemplary multimodal sensor 240b is depicted. Multimodal sensor 240b comprises at least one thermal sensor 226 positioned at the end of optical fiber 222. The at least one thermal sensor 226 each contain a temperature-sensitive material, wherein the temperature-sensitive material, including resistance material, is a phosphor, wherein temperature is measured by interrogating the material such as phosphor with near-infrared light and observing the decay frequency response. In certain embodiments, multimodal sensor 240b comprises a filter for 980 nm light integrated into the receiver electronics to reduce cross-talk from laser fiber 212.

The multimodal sensors comprise thermal sensors that are immune to electromagnetic interference, highly flexible, and resistant to self-heating, which reduces measurement error during hyperthermia. Typical performance is on the order of ±0.5° C. over a 50° C. range (35-85° C.), and ±2° C. over a temperature range of 0-120° C. Preferably, the multimodal sensors are constructed from optically transparent material that is thermally stable at or near temperatures typically encountered in FLA, such as in the range of 0-120° C. (e.g., Tefzel). The multimodal sensors can be under 1.5 mm in diameter, capable of fitting inside of a 15 Ga catheter for atraumatic insertion.

### Multi-Channel Needle Guide

In another aspect, the present invention provides a novel multi-channel needle guide. The multi-channel needle guide provides a platform for guided insertion of instruments such as laser fibers and sensors. The multi-channel needle guide comprises precise dimensions for the purpose of calculating laser coverage and treatment progress.

Referring now to FIG. 5, an exemplary multi-channel needle guide 300 is depicted. Multi-channel needle guide 300 comprises an elongate body having a first channel 302, at least one auxiliary channel 304, and a plurality of attachment clips 306. In some embodiments, multi-channel needle guide 300 further comprises locking member 308.

First channel 302 comprises first channel centerline 310. First channel 302 has a lumen that is sized to accept medical instruments suitable for use with the scope of the present invention. For example, in some embodiments, first channel 302 has a lumen that is sized to fit a biopsy needle, a catheter, a laser fiber, or a trocar. First channel 302 can have any suitable length. In some embodiments, first channel 302 has a length that is the same or less than the length of a typical ultrasound probe, such as a length between 5 and 15 cm.

Auxiliary channel 304 comprises auxiliary channel centerline 312. Auxiliary channel 304 also has a lumen that is sized to accept medical instruments suitable for use within the scope of the present invention. For example, in some embodiments, auxiliary channel 304 has a lumen that is sized to fit an optical sensor, a thermal sensor, or a multi-modal sensor. Auxiliary channel 304 can have any suitable length. In some embodiments, auxiliary channel 304 has a length between 5 and 15 cm. In some embodiments, auxiliary channel 304 has the same length as first channel 302, while in other embodiments, auxiliary channel 304 has a different length than first channel 302.

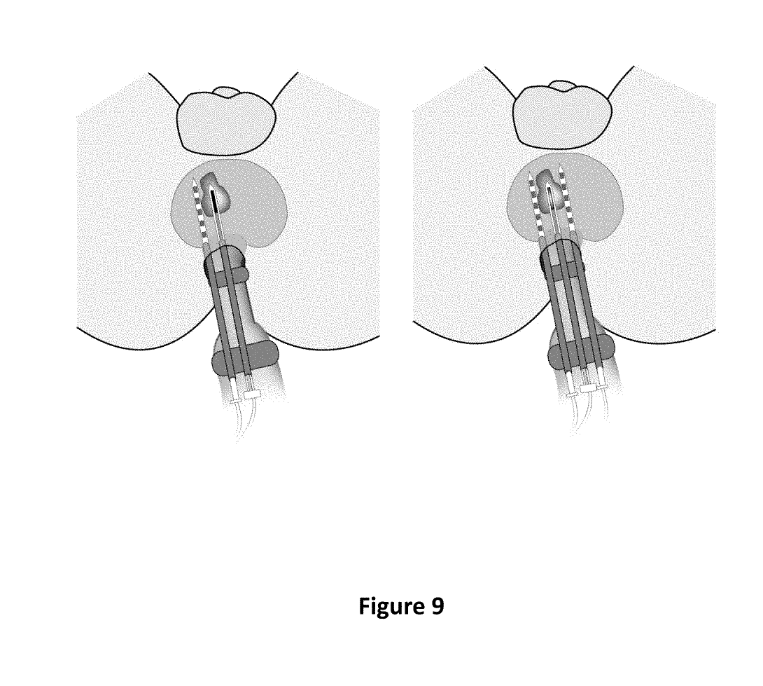

In some embodiments, multi-channel needle guide 300 further comprises at least one additional channels. Referring now to FIG. 9, a series of procedures relating to the treatment of the prostate illustrates the use of a multi-channel needle guide having two channels and a multi-channel needle guide having three channels.

As depicted in FIG. 5, multi-channel needle guide 300 may be attached to any suitable ultrasound probe, such as ultrasound probe 260. A plurality of attachment clips 306 may extend from first channel 302 and auxiliary channel 304 to secure multi-channel needle guide 300 to ultrasound probe 260. Attachment clips 306 may comprise features that enhance the fit between multi-channel needle guide 300 and ultrasound probe 260, such as tabs, hooks, slots, and the like. In certain embodiments, locking member 308 is provided to enhance the security of attachment. Locking member 308 can be any suitable locking mechanism that can be engaged to secure attachment and disengaged for detachment, such as a screw, a clamp, a bolt, a pin, and the like.

Multi-channel needle guide 300 comprises a range of specific dimensions to facilitate the processing of data detected by the instruments used in conjunction with multi-channel needle guide 300. Referring now to FIG. 5, certain dimensions relate to the distances between first channel 302 and auxiliary channel 304. Lateral distance 318 is the horizontal distance between first channel centerline 310 and auxiliary channel centerline 312. In some embodiments, lateral distance 318 can be between 1 and 20 mm. Vertical distance 320 is the height difference between first channel centerline 310 and auxiliary channel centerline 312. In some embodiments, vertical distance 320 can be between 1 and 2 mm.

As depicted in FIG. 5, other dimensions relate to the distances between first channel 302, auxiliary channel 304, and ultrasound probe 260. The point of reference for ultrasound probe 260 is transducer centerline 314. Vertical distance 322 is the height difference between first channel centerline 310 and transducer centerline 314. In some embodiments, vertical distance 322 can be between 10 and 15 mm. Vertical distance 324 is the height difference between auxiliary channel centerline 312 and transducer centerline 314. In some embodiments, vertical distance 324 can be between 10 and 14 mm.

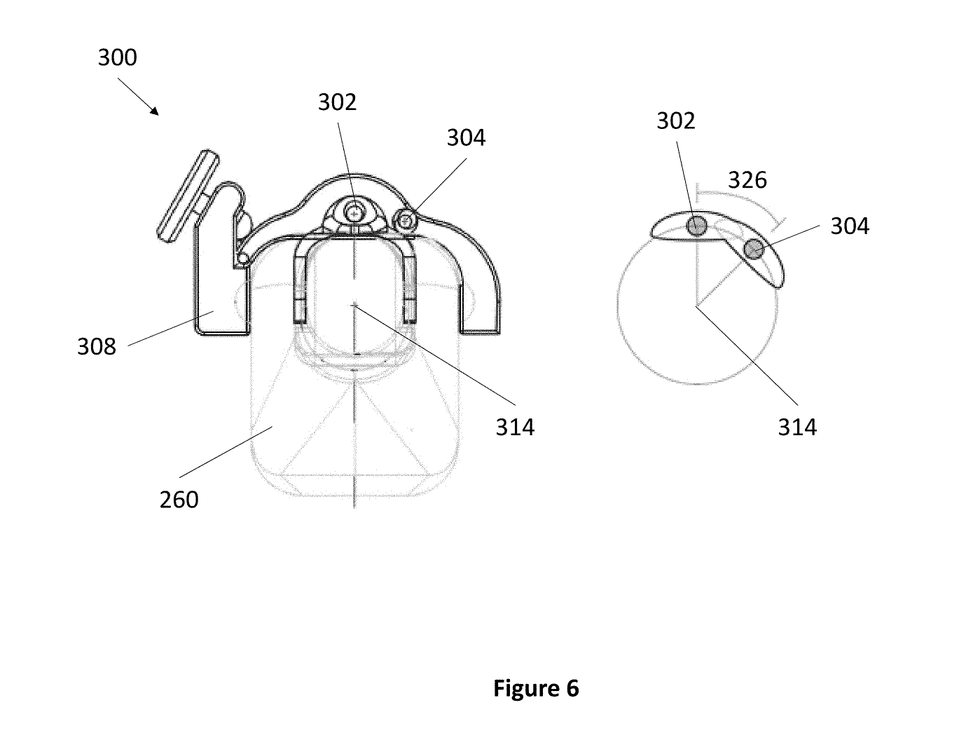

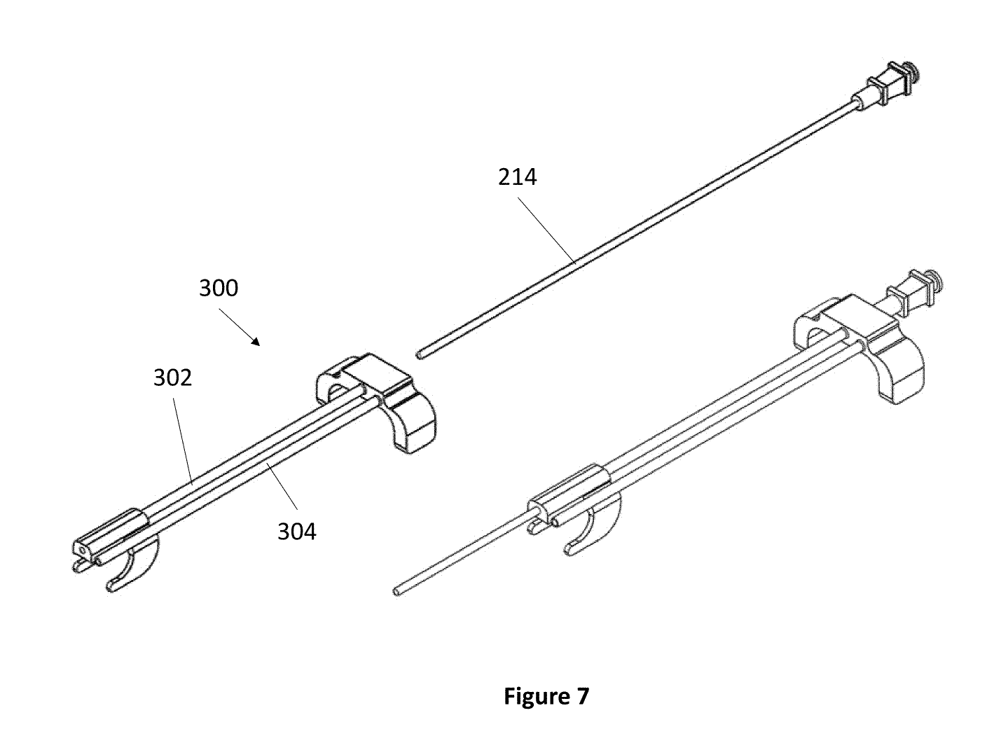

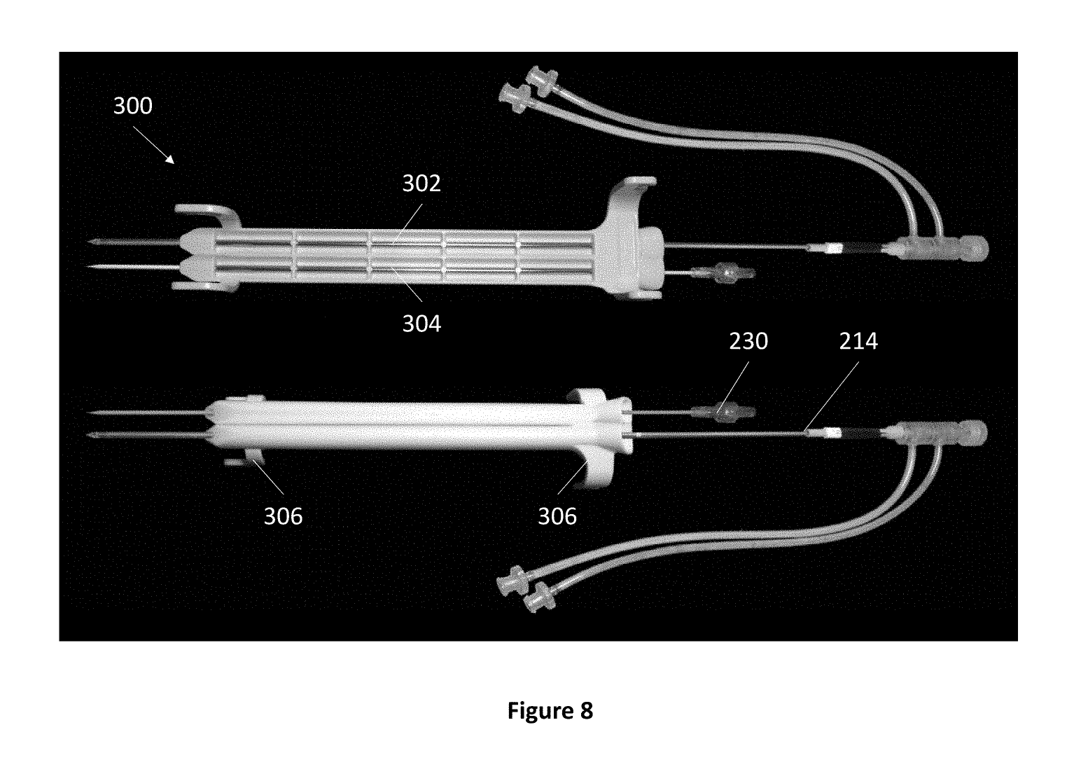

Referring now to FIG. 6, an alternative method of describing the distance between first channel 302, auxiliary channel 304, and ultrasound probe 260 is provided. From an anterior perspective, transducer centerline 314 represents the center of a circle, and first channel centerline 310 and auxiliary channel centerline 312 are positioned along the circumference of the circle, each having the same distance from transducer centerline 314. The distance between first channel centerline 310 and auxiliary channel centerline 312 can then be described as arc 326. In some embodiments, arc 326 can have a length between 1 and 20 mm.

Multi-channel needle guide 300 may comprise any suitable material, such as a plastic, a metal, or a composite material. Preferably, multi-channel needle guide 300 comprises a non-allergenic material. In some embodiments, multi-channel needle guide 300 comprises at least one label listing the exact measurements of the abovementioned dimensions. In some embodiments, the exact measurements are printed directly onto multi-channel needle guide 300. In some embodiments, the exact measurements are stored in a barcode, RFID chip, or other medium that is amenable to scanning for information transfer.

### Method of Focal Laser Therapy of Soft Tissue

In another aspect, the present invention provides a method of out-of-bore focal laser therapy of soft tissue using the systems and devices provided herein. The method is an improvement over the prior art in that it does not rely on MRI thermometry and can be performed in outpatient settings. The method uses ultrasound, optical sensors, and temperature sensors for real-time monitoring of treatment progress, reducing the time and cost of treatment.

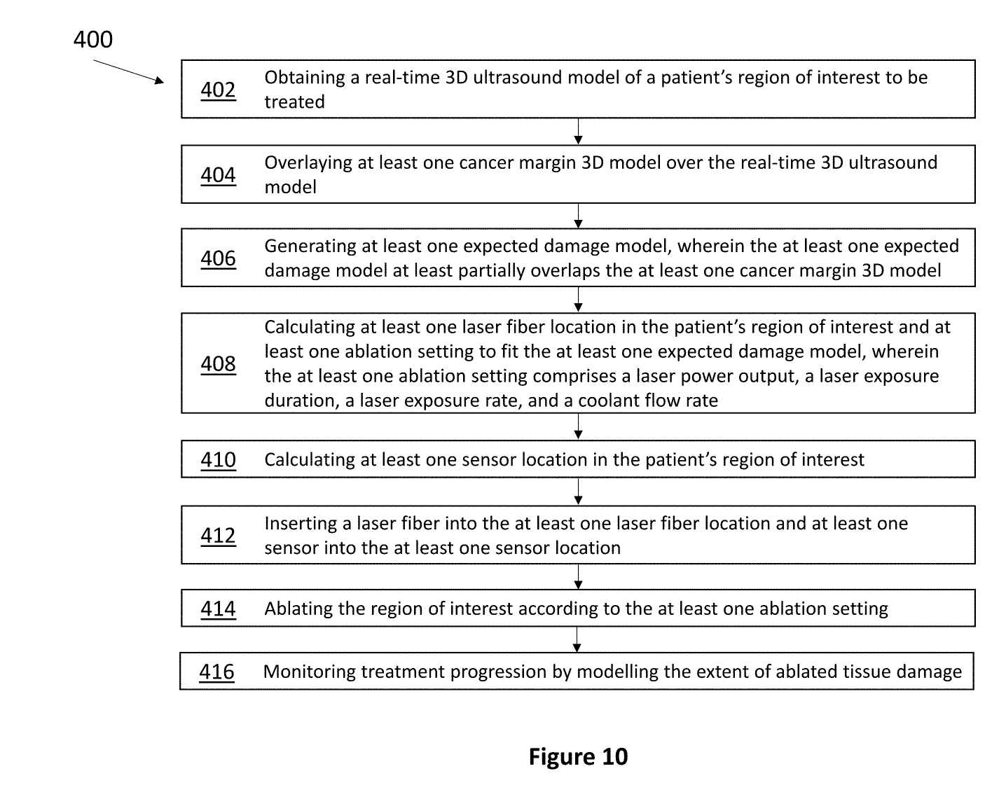

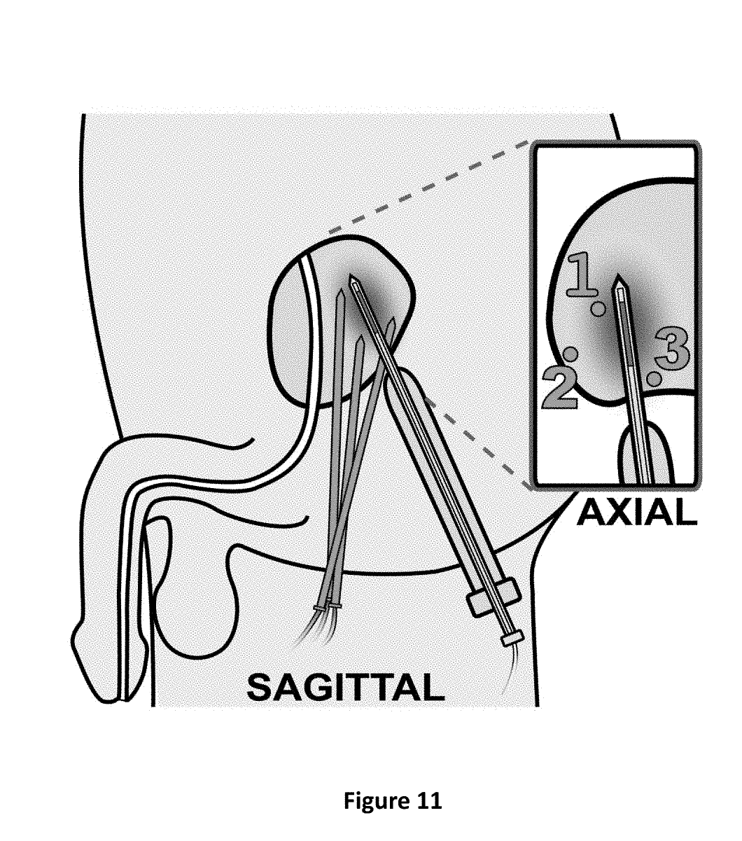

Referring now to FIG. 10, an exemplary method 400 of focal laser therapy of soft tissue is depicted. Method 400 begins with step 402, wherein a real-time 3D ultrasound model of a patient's region of interest to be treated is captured. In step 404, at least one cancer margin 3D model is overlaid on the real-time 3D ultrasound model. The at least one cancer margin 3D model comprises the 3D models generated from the method of cancer margin determination previously described herein: the MRI-visible lesion 3D model, the MTV 3D model, the Optimized Margin 3D model, and biopsy core information. In step 406, at least one expected damage model is generated, wherein the at least one expected damage model at least partially overlaps the at least one cancer margin 3D model. In step 408, at least one fiber location in the patient's region of interest is calculated, and at least one ablation setting to fit the at least one expected damage model is calculated, wherein the at least one ablation setting comprises a laser power output, a laser exposure duration, a laser exposure rate, and a coolant flow rate. In step 410, at least one sensor location in the patient's region of interest is calculated. In step 412, a laser fiber is inserted into the at least one laser fiber location, and at least one sensor is inserted into the at least one sensor location. In step 414, the at least one ablation setting is executed. In step 416, treatment progression is monitored by modelling the extent of tissue damage.

A real-time 3D ultrasound model of a patient's region of interest to be treated is captured using a needle guide (such as multi-channel needle guide 300) attached to ultrasound probe 260 and 3D scanning and location tracking assembly 270. In some embodiments, ultrasound probe 260 is rotated to scan at a plurality of angles to generate the 3D ultrasound model. The real-time 3D ultrasound model is transmitted and displayed on computer platform 280.

Computer platform 280 combines the real-time 3D ultrasound model with the at least one cancer margin 3D model (the MRI-visible lesion 3D model, the MTV 3D model, the Optimized Margin 3D model, and biopsy core information). Computer platform 280 overlays the cancer margin 3D models over the patient's real-time 3D ultrasound model using multi-modal image fusion, including elastic registration, and creates a treatment plan comprising laser fiber positioning, sensor positioning, laser power output, and laser activation time.

In one embodiment, computer platform 280 may base its treatment plan off of an ablation setting wherein a laser fiber emitting 13.75 W for 3 minutes at a high coolant flow rate causes coagulative necrosis to surrounding tissue that is ellipsoidal in shape and has a volume of approximately 4 cc. Variability in thermal conductivity and vasculature in the surrounding tissue produces an expected damage model comprising 3 nested ellipsoids, wherein the smallest ellipsoid represents minimum expected damage (minED), the middle ellipsoid represents average expected damage (aveED), and the largest ellipsoid represents maximum expected damage (maxED) at a given set of ablation settings, with the laser fiber at the center of the ellipsoids.

Computer platform 280 enables an operator to overlay an expected damage model over the MRI-visible lesion 3D model, the MTV 3D model, the Optimized Margin 3D model. The operator may freely manipulate the expected damage model, such as by changing spatial location, orientation, and scale, such that the expected damage model encapsulates cancer harboring tissue as indicated by the aforementioned 3D models. In some embodiments, the operator may overlay a plurality of expected damage models to better capture all cancer harboring tissue. For instance, if the cancerous tissue is oblong in shape or present in more than one location, an operator may overlay more than one expected damage model. At a minimum, the minED of the expected damage model must fully encapsulate the volume of the MTV 3D model, or else there will be a chance of leaving cancerous tissue untreated.

In some embodiments, computer platform 280 comprises a monitoring and alert system that detects the overlap of an expected damage model with a sensitive anatomical feature. For example, if computer platform 280 detects that the operator has placed an expected damage model that will cause unacceptable damage to a sensitive structure such as the rectal wall, an alert may sound. In some embodiments, computer platform 280 comprises a deformation algorithm that automatically modifies the treatment plan to account for anatomical features.

Computer platform 280 uses the expected damage models placed by the operator to assess the likelihood of destroying all cancer by comparing the expected damage model volume with the Optimized Margin 3D model, as well as by reporting the likelihood of damaging sensitive anatomy that is in close proximity. Based on the assessment provided by computer platform 280, an operator may amend the expected damage model placement until an acceptable treatment plan is reached.

Upon confirmation that the treatment plan is acceptable, computer platform 280 calculates ideal laser fiber positions and temperature, optical, or multi-modal sensor positions to fit the expected damage models, and appropriate angle of insertion to minimize damage to sensitive anatomy. In some embodiments, an operator uploads the dimensions of the multi-needle channel guide into computer platform 280 to facilitate the calculations. The operator may optionally direct computer platform 280 to include additional sensors, which is advantageous when temperature monitoring is desired at additional locations.

Computer platform 280 also calculates ideal ablation settings (laser power output, laser exposure duration, laser exposure rate, and coolant flow rate) to fit the expected damage models. The operator will be provided with a range of the expected temperatures according to ablation settings. The operator can approve the expected temperatures, or reject and manually adjust the ablation settings. The maximum allowable temperature indicates the upper limit of temperature at the probe locations before tissue vaporization is risked at the center of heating. Preferably, the maximum temperature is less than 95° C. The minimum temperature is the minimum temperature at a probe location that is required to achieve the level of coagulative necrosis necessary to fit the expected damage models. If the operator included additional thermal sensors, maximum temperatures may also be set for each additional thermal sensor. Upon reaching the maximum temperature, computer platform 280 may reduce or shut off laser output to prevent further damage.

Computer platform 280 transmits the calculated positions and appropriate angles of insertion of the laser fiber and sensors to 3D scanning and location tracking assembly 270. 3D scanning and location tracking assembly 270 is used to move the multi-channel needle guide 300 and ultrasound probe 260 into position. In some embodiments, an echogenic trocar is inserted through the first channel 302 of the multi-channel needle guide 300 and into the patient, a dual lumen catheter is inserted through the echogenic trocar into the patient, then the laser fiber is inserted through a lumen of the dual lumen catheter. An optical sensor 220, thermal sensor 230, or multi-model sensor 240 is inserted through the auxiliary channel 304 of the multi-channel needle guide 300 and into the patient. The positions of the laser fiber and sensor are tracked by the real-time 3D ultrasound model to confirm correct placement.

Computer platform 280 transmits the calculated ablation settings to laser 210, and the operator may initiate the focal laser therapy. While the ablation settings calculated by computer platform 280 are recommended, the operator is free to modify the ablation settings. In some embodiments, the operator may initiate a test burn prior to applying the full treatment dose, wherein the laser fiber is activated at low power to interrogate the treatment plan parameters.

Treatment progress is monitored by modelling the extent of coagulative necrosis based on measurements provided by the optical, thermal, or multi-modal sensors. In some embodiments, treatment progress can be monitored using a thermal damage model. The at least one thermal sensor placed near the expected damage volume records temperature in real-time and computer platform 280 uses the temperature and positional information to extrapolate the temperature throughout the expected damage volume to estimate the extent of coagulative necrosis.

In some embodiments, treatment progress can be monitored using treatment induced alteration of thermal properties. The theory behind treatment induced alteration of thermal properties is that destroying cancerous tissue should also disrupt its vascular network. Treatment induced alteration of thermal properties therefore examines change in tissue perfusion as a means of modelling coagulative necrosis. If the vascular network has been successfully disrupted by the treatment, then the rate of tissue cooling is expected to decrease significantly. The change in perfusion may be observed by performing a test burn at low power and using at least one thermal sensor to measure the rate of tissue cooling, then performing the full treatment burn and measuring the rate of tissue cooling immediately after the full treatment burn.

In some embodiments, treatment progress can be monitored by measuring changes in ultrasound images. Various ultrasound imaging techniques may be used to estimate tissue damage, including but not limited to: measuring changes in tissue temperature, mechanical properties, and vascularity. In some embodiments, contrast agents such as microbubbles can be used to detect changes in perfusion rate to estimate the level of coagulative necrosis in an image region, either during or after laser application.

In some embodiments, treatment progress can be monitored by quantifying thermally induced alterations in tissue optical properties. The theory behind treatment induced alteration of optical properties is that thermally induced changes in tissue proteins correlate well with tissue optical properties. An optical monitoring system is also capable of providing real-time volumetric information. The propagation of light in tissue is governed by the absorption coefficient (μa) and the reduced scattering coefficient (μs′). An increase in either of these results in an increase in attenuation of light. Studies have shown that thermally induced tissue damage can cause up to a three-fold increase in total attenuation (Jaywant S et al., Laser-Tissue Interaction 1882, (1993):218-229; Nau W H et al., Lasers Surg. Med. 24, (1999):38-47). Therefore, radiance measured by an optical probe placed at a distance from an interstitial laser fiber will decrease as the tissue coagulates (Whelan WM et al., Int. J. Thermophys. 26, (2005):233-241). In contrast to the commonly used thermal monitoring systems, an optical approach does not rely on dose models to estimate coagulation. The change in tissue optical properties may be observed using at least one optical sensor.

In some embodiments, treatment progress can be monitored using one or more of the abovementioned models. In certain embodiments, multi-modal sensors comprising at least one optical sensing element and at least one thermal sensing element may be used to monitor treatment progress using one or more of the abovementioned models.

After treatment in a laser fiber location, computer platform 280 provides a probability of treatment success based on the accumulated laser energy, time, location, and treatment plan. If the thermal changes in the tissue alter the initial expected damage models, computer platform 280 may dynamically update the treatment plan to fit the new expected damage models. The operator may repeat the relevant steps of positioning and inserting the laser fiber and sensors, administering treatment, and updating the expected damage models until total coverage of the expected damage volume is complete.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out the preferred embodiments of the present invention, and are not to be construed as limiting in any way the remainder of the disclosure.

### Example 1: Focal Laser Ablation of Prostate Cancer: Feasibility of MR/US Fusion for Guidance

Focal laser ablation (FLA), or laser interstitial thermal therapy (LITT), is a method for treating prostate cancer without surgery or ionizing radiation (Bomers JGR et al., World Journal of Urology (2016): 1-9). The goal of FLA is to induce coagulation necrosis of cancerous prostate tissue by use of an interstitially placed diffusing laser fiber (Lee T et al., Reviews in urology 16.2 (2014); Stafford R J et al., The Journal of urology 184.4 (2010): 1514-1520). FLA was first described for prostate treatment in 1993 (Johnson D E et al., Lasers in surgery and medicine 14.4 (1994): 299-305) and has been the subject of a number of recent investigations (Oto A et al., Radiology 267.3 (2013): 932-940; Natarajan S et al., The Journal of urology 196.1 (2016): 68-75

Lepor H et al., European urology 68.6 (2015): 924-926; Eggener S E et al., The Journal of urology 196.6 (2016): 1670-1675; Lindner U et al., Journal of Endourology 24.5 (2010): 791-797). The procedure appears safe and feasible, and because the core technology is FDA-approved it is being offered commercially (Lepor H et al., European urology 68.6 (2015): 924-926).

As currently performed, FLA is accomplished within the gantry of an MRI scanner (in-bore), and the operator is a radiologist (Oto A et al., Radiology 267.3 (2013): 932-940; Natarajan Set al., The Journal of urology 196.1 (2016): 68-75; Lepor H et al., European urology 68.6 (2015): 924-926). In-bore FLA allows direct targeting of a cancerous region by MRI guidance and also allows monitoring of temperature changes in the prostate via MR thermometry (Nour S G, Seminars in interventional radiology. Vol. 33. No. 03. Thieme Medical Publishers, 2016). In a preliminary study, these features were confirmed but the procedure was found to be lengthy, expensive, and resource-intensive (Natarajan S et al., The Journal of urology 196.1 (2016): 68-75).

In the following study, the safety and feasibility of simplifying FLA was evaluated by performing the procedure in a clinic setting (out-of-bore) instead of within an MRI scanner. An extensive in-house experience with MRI/US fusion for biopsy targeting (Sonn G A et al., The Journal of urology 189.1 (2013): 86-92) provided the impetus for using a similar approach for targeted treatment. Previous use of thermal probes for intra-prostatic temperature monitoring during in-bore FLA demonstrated that monitoring out-of-bore FLA was possible (Natarajan S et al., The Journal of urology 196.1 (2016): 68-75). Therefore, FLA was performed in the urology clinic using MRI/US fusion and interstitial thermal probe monitoring. In keeping with the simplicity aim, only local anesthesia and minimal sedation were used.

The materials and methods are now described.

### Patients

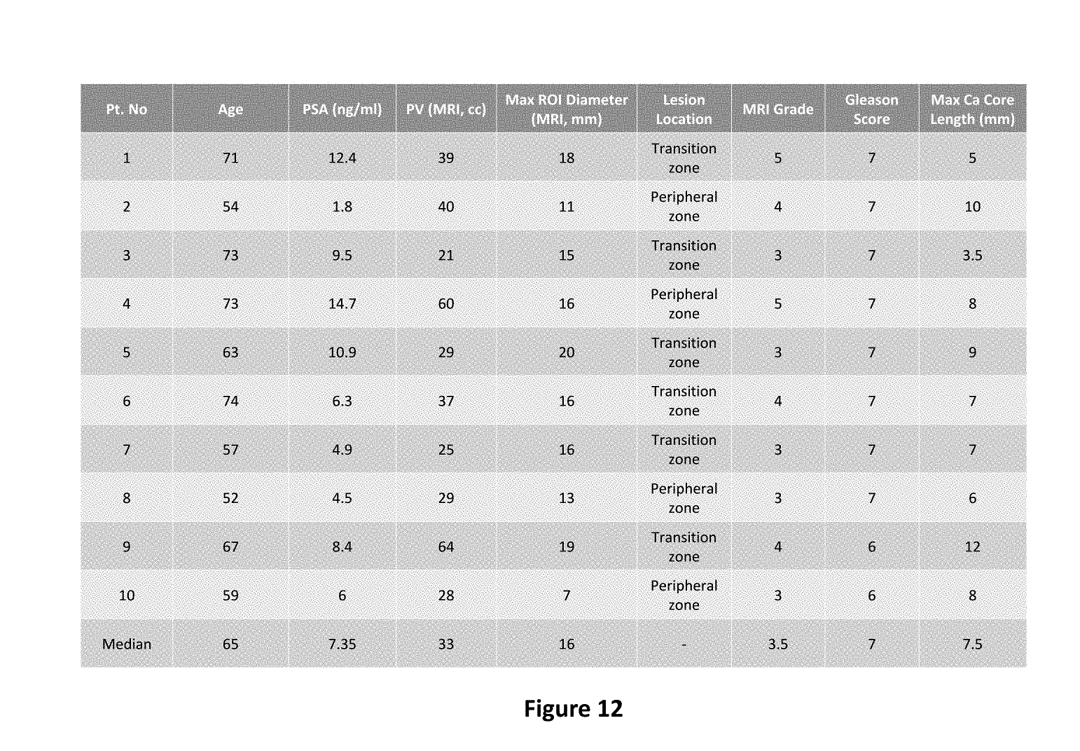

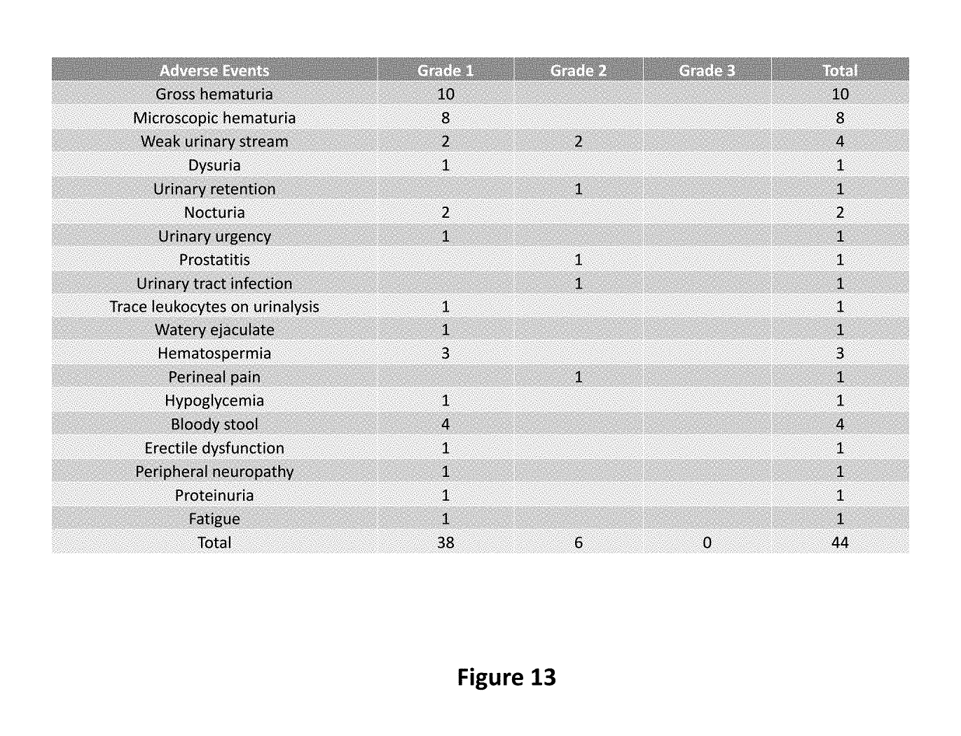

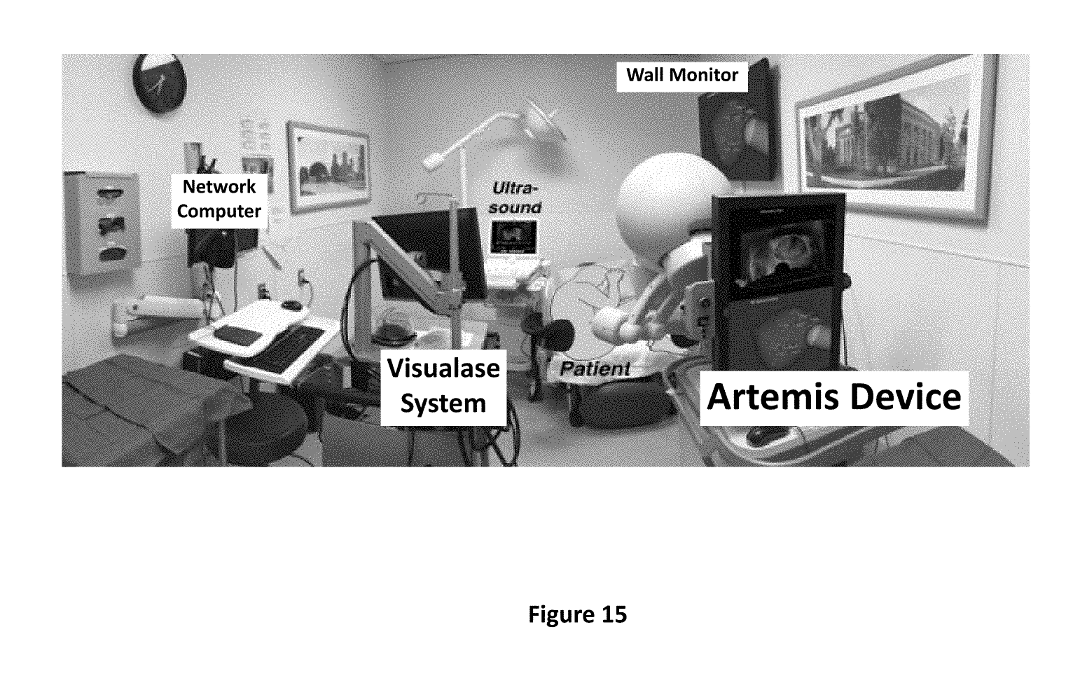

Men with intermediate-risk prostate cancer were subjects of this study. In each case, the cancer had been confirmed by MRI/US biopsy to be present only within an MRI-visible region of interest (ROI). MRI and biopsy procedures were as described previously (Sonn G A et al., The Journal of urology 189.1 (2013): 86-92). Inclusion and exclusion criteria are shown in FIG. 12. The primary endpoint was absence of any treatment-related grade 3 or greater adverse events (CTCAE, v4.03) during a 6-month follow-up period. Exploratory endpoints were lack of decline in urinary and sexual function, PSA decline, and change in histology or MR imaging. FLA was performed using a room set-up similar to that employed for fusion biopsy (FIG. 15). Patient characteristics are shown in FIG. 13.

### Procedure Planning

Each patient received targeted biopsy via the Artemis device prior to enrollment in this study, which allows for storage of the location of each ROI and biopsy core in 3D. Each patient's imaging and biopsy information was used to plan each treatment. Treatment margins were intentionally kept conservative in this early-phase study.

### Treatment Protocol

All men were given a cleansing enema and antibiotic prophylaxis with an oral quinolone and an injection of ceftriaxone or ertapenem. Just prior to the procedure, all patients received a single intravenous dose of ketorolac (30 mg) and midazolam (4 mg) (minimal sedation). Patients were placed in the left lateral decubitus position for transrectal US and periprostatic nerve block using a 50-50% mixture of bupivacaine and lidocaine. Following periprostatic anesthesia, patients were turned into lithotomy position for perineal insertion of thermal probes. Using intra-cutaneous lidocaine (1%), 2 to 3 MR-compatible fluoroptic temperature probes (STB, LumaSense, Santa Clara, Calif.) were placed trans-perineally into the prostate using real-time ultrasound guidance. At least 1 probe was advanced into the posterior prostate near the rectal wall for intra-prostatic temperature monitoring as described elsewhere (Natarajan S et al., The Journal of urology 196.1 (2016): 68-75). Vital signs were monitored continuously, and pain scores (Hawker G A et al., Arthritis care & research 63.S11 (2011): S240-S252) were numerically assessed prior to, during, and following each laser activation.

After thermal probe placement, patients were returned to the lateral decubitus position for reinsertion of the ultrasound probe and attachment of the probe to the fixed tracking arm of the Artemis fusion device (Eigen, Grass Valley, Calif., USA) shown in FIG. 16. Pre-planned laser fiber positions and the pre-operative MRI were loaded into the device and fused with real-time ultrasound.

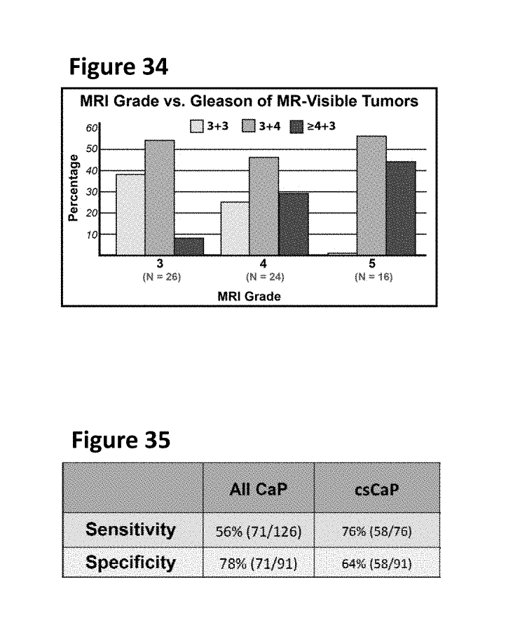

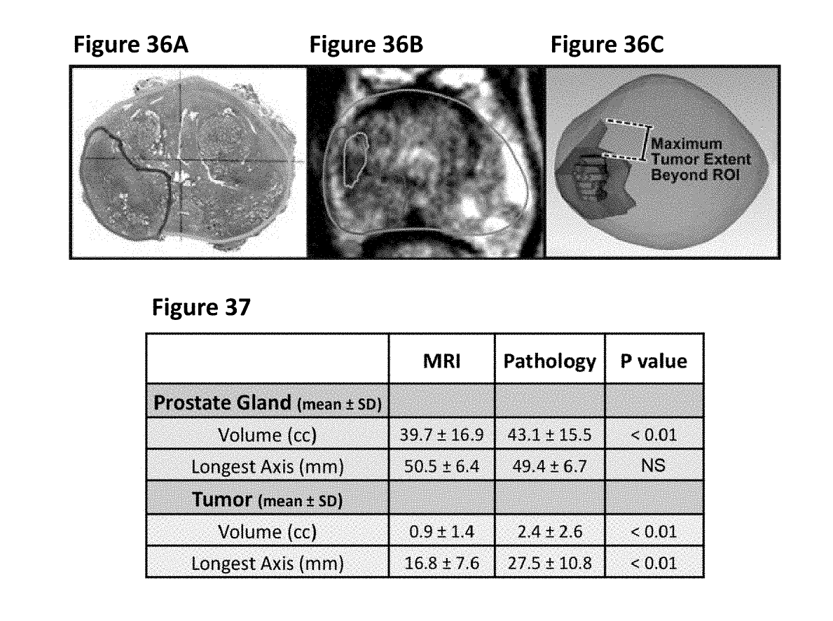

For insertion of the laser fiber, a needle guide was fabricated which included a channel for a laser fiber and a parallel channel for a thermal probe. This needle guide enabled placement of a temperature probe parallel to the laser fiber for direct intra-prostatic temperature monitoring for treatment efficacy. Treatments were monitored using the temperature of each probe, ultrasound information, and the flow-rate of the cooling pump. FIG. 17 displays an example of the spatial relationship of the laser fiber and probes in the prostate during FLA.

Components of an existing MRI-guided FLA system (Visualase, Medtronic) were adapted for this new procedure, including a 15 W 980 nm laser (Biotex) and surgical infusion pump (K-pump, KMI). Real-time ultrasound was used to guide the biopsy needle tip to the ROI. The needle was replaced with a dual lumen catheter that contains the laser fiber (Uro-kit 600, Medtronic) and circulates saline for active cooling. The fixed arm of the Artemis fusion device provided a stable platform for securing and when necessary, repositioning of the laser fiber during the procedure.

Several laser activations of 13.75 W for 1-3 minutes were used in each treatment. If a difference between the a priori plan and real-time ultrasound image was observed, i.e. mis-registration, the prostate was re-scanned and the segmentation and registration procedures were repeated. Thermal probes provided continuous monitoring of intra-prostatic temperature throughout the procedure. Laser application was manually ceased if rectal wall temperatures exceeded 42° C.

Prior to termination of each procedure, three fiducial markers were implanted into the prostate to provide a reference for follow-up imaging. After a one-hour observation period, patients underwent repeat mpMRI. Dynamic Contrast Enhancement (DCE) MRI was used to confirm non-perfusion of the ablated zone. All patients were discharged home with a quinolone antibiotic and oral non-narcotic analgesics within 1 to 2 hours following FLA.

### Follow-up Evaluations

Follow-up clinic visits were conducted at 1 week, 1 month, 3 months and 6 months. Each visit included a detailed history and physical examination, screening for adverse events, medication reconciliation, PSA, and health-related quality of life (HRQOL) questionnaires (e.g., IPSS, IIEF). 3T mpMRI was performed at 6 months and interpreted using PI-RADS scoring and criteria developed at UCLA (11,13). At 6-month biopsy, MRI/US fusion (Artemis) was used to sample the original tumor site, the ablation zone, margins of the ablation zone, any new ROI, and six template sites throughout the ipsilateral prostate. An average of 12 biopsy cores (range 9 to 16) were obtained from the treated side of each prostate.

The results are now described.

Eleven men were enrolled, and FLA was successfully performed in 10. Summaries of each patient are given in FIG. 13. In one patient, FLA was aborted prior to initial laser activation; in this individual, the combination of a large TURP defect and a small prostate (15 cc) precluded secure anchoring of the fiber within the prostate, and treatment was not attempted. Among the 10 patients treated, mean procedure time was 95 minutes (range 71 to 105). After the first several patients, the procedure was modified to include an echogenic introducer needle to improve US localization of the laser fiber.

The laser fiber was activated a median of 5 times at a power of 13.75 W for an average of 144 seconds during each procedure. To ensure complete treatment of the ROI, the laser fiber was re-positioned an average of 2 times for each patient.

### Adverse Events

Thirty-eight grade 1 and six grade 2 adverse events were recorded during a 6-month period. One patient was hospitalized 2 months after FLA for elective surgical correction of pre-existing lumbar stenosis, which was planned prior to study enrollment. Hematuria was the most frequent adverse event following treatment and resolved in all patients without intervention. All patients were discharged home from the outpatient clinic procedure room after treatment and follow-up MRI. No treatment-related serious adverse events (>Grade 3, CTCAE) were encountered.

### Clinical Effects of FLA

HRQOL questionnaires were performed at 1 week, 1 month, 3 months and 6 months following FLA. The median IPSS at baseline was 7 and decreased to 5.5. The median IIEF-5 score at baseline was 14 and increased to 19. No change was significant. Median PSA at baseline was 7.35 ng/ml at baseline and decreased to 2.55 ng/ml at 6 months (Wilcoxon signed-rank test p=0.28).

### Temperature Data

Data from fluoroptic thermal probes were successfully recorded in all patients. An average of 2 transperineal probes were inserted into the prostate in addition to the transrectal monitor. Maximum temperature recorded near the tip of the laser fiber was 68° C. If the rectal monitor approached 42° C., laser activation was stopped and the fiber repositioned within the prostate. In all cases the thermal probe closest to the rectum recorded temperatures below 42° C.

### MRI Changes

Immediately after the procedure, MRI revealed a confined, localized hypo-perfusion of the treated area, i.e., an ablation zone, in each patient (FIG. 19). Median volume of the ablation zone as determined by MRI was 4.8 cc. No major treatment-related changes in T2 or diffusion weighted imaging was seen. Median prostate volume did not change significantly from pre- to 6 months post-treatment (33 vs. 32 cc, p=0.44, Wilcoxon signed-rank test).

### 6-month Biopsy Results

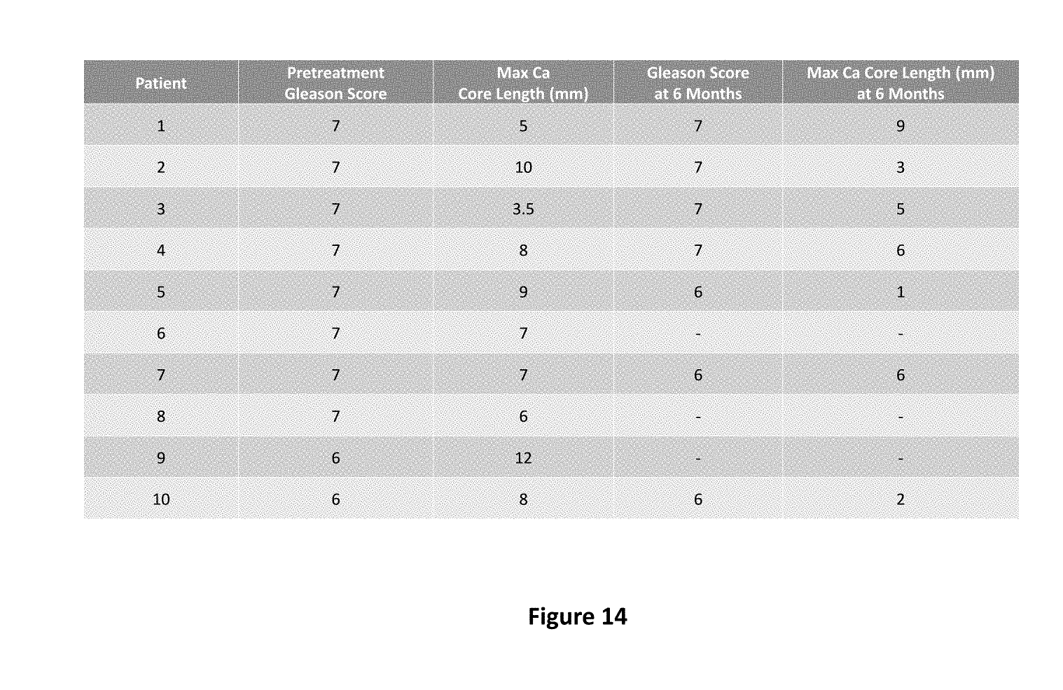

Results of follow-up biopsies were related to operator experience of FLA and addition of an echogenic needle. In the first four patients, biopsy revealed continued presence of clinically significant disease in both the treatment zone and margin. In the next six patients, biopsy revealed micro-focal Gleason 3+3 disease in 3 (1 within treatment zone, 2 in margin), and complete absence of cancer in the other 3 men (FIG. 14). Biopsy material from the treatment zone often revealed benign prostate glands and stroma with chronic inflammation, hemosiderin-laden macrophages, giant cell reaction and stromal fibrosis consistent with thermal effect. FIG. 20A through FIG. 20F depict such findings in one patient.

Focal therapy is an emerging alternative to whole-organ CaP treatment that promises localized cancer control without treatment-related adverse events frequently seen with other modalities. Improvements in prostate MRI have allowed focal therapy to become a more viable option in CaP treatment (Cepek J et al., Medical physics 41.1 (2014)). Recent evidence suggests that focal therapy is a safe approach to CaP treatment (Oto A et al., Radiology 267.3 (2013): 932-940; Natarajan S et al., The Journal of urology 196.1 (2016): 68-75; Lepor H et al., European urology 68.6 (2015): 924-926). In a recent systematic review, Valerio et. al reported favorable rates of continence (95-100%) and erectile function (54-100%) following focal therapy (Valerio M et al., European urology 66.4 (2014): 732-751). However, there is a paucity of long-term clinical data regarding cancer control and HRQOL outcomes. A recent workshop hosted by the FDA, American Urological Association, and Society of Urologic Oncology stated that “currently available technologies are capable of selective ablation of the prostate gland with reasonable accuracy, but that criteria for the selection of patients appropriate for PGA remain debatable” (Jarow J P et al., Urology 88 (2016): 8-13).

In the present study, out-of-bore FLA was found to be technically feasible and safe for the treatment of intermediate risk CaP in an outpatient clinic. The study differs from others in that FLA was performed without direct MRI guidance and in the treatment room of a urology clinic. Guidance and targeting was achieved using MRI/US fusion, and temperature monitoring was achieved using thermal probes. Lindner et. al. previously performed FLA using MRI/US fusion guidance in patients with low risk CaP (Lindner U et al., The Journal of urology 182.4 (2009): 1371-1377). However, the Lindner procedures were performed trans-perineally and required general anesthesia (Lindner U et al., The Journal of urology 182.4 (2009): 1371-1377). In the current study, all patients were treated under local anesthesia with only minimal sedation and discharged home 1-2 hours following treatment. No grade 3 or greater adverse events were observed; urinary and sexual function remained intact.

In a Phase 2 study, Eggener et al from University of Chicago found that in-bore FLA produced encouraging oncologic outcomes (Eggener S E et al., The Journal of urology 196.6 (2016): 1670-1675). In that study, targeted biopsy of the ablation zone 3 months after treatment revealed persistent cancer in only 1/27 men (Eggener S E et al., The Journal of urology 196.6 (2016): 1670-1675). At 12 months, systematic biopsy revealed cancer in 10 men (37%). While the Chicago results appear superior to the present results, the studies are not comparable. In the present study, safety and feasibility were the primary outcomes of interest, because of the nearly unprecedented out-of-bore approach. Regarding oncologic outcomes, tumors in the present trial were intermediate-risk compared to mostly low-risk in the Chicago trial. Also, biopsy was more extensive in the present trial than in the earlier work. Despite the above differences, the ablation volumes were similar in both studies, and also comparable to our own in-bore results (Natarajan S et al., The Journal of urology 196.1 (2016): 68-75). Importantly, the reported safety results are the same in-bore versus out-of-bore, without serious adverse events in either approach. Thus, performance of focal laser ablation—out-of-bore, in a urology clinic under local anesthesia—appears feasible and safe. Moreover, the out-of-bore treatment promises to be relatively inexpensive, quick, and efficient.

In the present study, subjects were men with intermediate-risk CaP, modeled after a previous trial using both MR thermometry and fluoroptic thermal probes for temperature monitoring (Natarajan S et al., The Journal of urology 196.1 (2016): 68-75). Thermal probe data indicating procedure safety was the justification for using thermal probes alone for treatment monitoring. In a direct comparison, thermal probe recordings compared favorably to MRI thermometry for determination of intra-prostatic temperatures during FLA (Natarajan S et al., The Journal of urology 196.1 (2016): 68-75). The outcomes in this study were similar to the previous study including safety, HRQOL, and treatment-related changes on imaging. However, mean procedure time was reduced as compared to the prior in-bore study from 292 minutes to 95 minutes (Natarajan S et al., The Journal of urology 196.1 (2016): 68-75).

### Example 2: Optical-Based Estimation of Tissue Damage

Radiance sensors have been shown to be more sensitive than fluence sensors to coagulation induced changes in tissue optical properties (Chin L C L et al., Optics Letters 29, (2004):959-961). Furthermore, Chin et al demonstrated that the radiance at 0 degrees (facing the light source) steadily decreases in signal as the coagulation zone develops. In contrast, the radiance at 180 degrees (facing away from the light source) increases in signal once the coagulation boundary passes the probe. The thermally induced coagulation causes an increase in scattering, and results in an increase in the back-scattered photons scattered towards the radiance sensor once the coagulation front passes the probe (Chin L C L et al., Optics Letters 29, (2004):959-961). In the following study, an integrated multimodal sensor is developed consisting of a thermal sensor and 2 radiance sensors facing in opposite directions. Such a probe would be capable of detecting both the coagulation boundary and char development around the fiber tip. Lasing parameters may be modulated to achieve the optimal ablation zone. This technique can be used to monitor any ablation modality including high intensity focused ultrasound and radiofrequency. It is particularly suitable for LITT as the laser inducing coagulation can also be used to monitor its progress. While the ability to monitor coagulation will be lost once the laser is deactivated, data shows that due to rapid cooling only minimal damage occurs at this stage (FIG. 21)

Studies were conducted in bovine muscle using the setup shown in FIG. 22. An optical monitoring system was developed in which an optical probe was inserted into the tissue via a catheter. It is strategically located at the periphery of the intended target. A photodiode was used to convert the interstitial light intensity to a photovoltage. This photovoltage is proportional to fluence or radiance. In the setup in FIG. 22, the probe is shown at a radial distance of 8 mm and is advanced further than the laser diffuser. This setup was chosen as both ex vivo experimental work and clinical trial data showed that the laser diffuser tends to emit light in a forward direction. Using a catheter, a fluoroptic temperature probe (Lumasense, Santa Clarita, Calif.) was placed diametrically opposite to provide a comparison. LITT was performed at 13.7 5W for 200 s.

Temperature and normalized photovoltage are shown in FIG. 23. Within seconds of laser activation, the temperature begins to rise while the normalized photovoltage falls. Normalized photovoltage falls because the tissue coagulates causing an increase in the reduced scattering coefficient and thus total attenuation. Interestingly after approximately 60 s the normalized photovoltage stops falling. This appears to indicate that the coagulation boundary is approaching the sensor. These events are not detected by the thermal sensor, which continues to show a steady rise in temperature.

FIG. 24 adds the damage estimates using the same parameters outlined earlier. Again the normalized photovoltage drops throughout the procedure indicating the development of tissue coagulation while none of the damage estimates show significant coagulation until 100 s. This clearly demonstrates that unlike the thermal system, the optical monitoring system provides an instantaneous representation of opto-thermal events occurring throughout the volume. Furthermore, the slope of the normalized photovoltage could be used to modulate laser power. For example, a steep slope may indicate that char will occur before the desired volume is ablated. This data could be used to decrease laser power; thus, allowing for greater heat transfer via conduction before the tissue chars. In this way the size of the ablation zone can be maximized. Char also causes damage to the laser fiber. Once the tissue is charred the fiber needs to be repositioned to continue treatment. Again the optical monitoring system can provide this information while a purely thermal system cannot.

### Example 3: Validation Using Prostate Phantom

In order to observe the propagation of the coagulation boundary in real-time, a phantom is developed that mimics the optical and thermal properties at 980 nm of prostate tissue. The phantom contains the specific heat capacity (3.779 J/(g*K)) and thermal conductivity properties (0.56 W/m/K) previously found for human prostate (Giering K et al., Thermochim. Acta 251, (1995):199-205; Van den Berg CaT et al., Phys. Med. Biol. 51, (2006):809-825). One half of the phantom will possess dynamic optical properties as outlined by Iizuka et al (Iizuka M N et al., Lasers Surg. Med. 25, (1999):159-169). Previously derived absorption and reduced scattering coefficients (Ws=8.1 cm', μa=0.66 cm') are used (Bu-Lin Z et al., Int. J. Hyperthermia 24, (2008):568-576). This ensures that during LITT the optical scattering properties will change as is observed in vivo. The remaining half of the phantom consists of optically transparent acrylamide. A high-speed camera is positioned on this side to record coagulation zone development as demonstrated by Zhang et al (Bu-Lin Z et al., Int. J. Hyperthermia 24, (2008):568-576). In their work the coagulated region was clearly demarcated as a white zone around the ablation applicator. A further approach is to add thermochromic ink which has been demonstrated as a useful method of examining the temperature profile during LITT (Mikhail A S et al., Med. Phys. 4304 (2016); Negussie A H et al., Int. J. Hyperthermia 6736 (2016):1-5). The laser diffuser and multimodal sensor are placed on the interface between the two halves of the phantom. The optical fibers and thermal probe in the multimodal sensor are connected to photodiodes and the temperature monitoring system respectively. This approach allows the correlation of the radius of coagulation with radiance to demonstrate that the radiance at 180 degrees increases as coagulation front passes the sensor due to increased back-scatter (Chin L C L et al., Optics Letters 29, (2004):959-961).

### Example 4: Focal Laser Ablation of Prostate Cancer

The advent of multi-parametric MRI (mpMRI) for localization of prostate cancer (CaP) and targeted biopsy has provided a scientific basis for focal therapy research (Ahmed H U et al., The Journal of Urology, 2011, 185(4):1246-1255; van den Bos W et al., Eur Radiol, 2015, 1-9; Lepor H et al., European Urology, 2015, Epub ahead of print; Oto A et al., Radiology, 2013, 267(3):932-940). Theoretically, focal therapy offers the possibility of cancer control with little treatment-related morbidity (Ahmed H U et al., The Lancet Oncology, 2012, 13(6):622-632), but only a few clinical trials have been performed to date. Ahmed et al. used high-intensity focused ultrasound (HIFU) to treat MRI-identified lesions in 42 men (Ahmed H U et al., The Lancet Oncology, 2012, 13(6):622-632). Oto and colleagues used focal laser ablation (FLA) to treat MRI-identified lesions in 8 men (Oto A et al., Radiology, 2013, 267(3):932-940). Van den Bos et al recently reported use of irreversible electroporation (IRE) to focally treat lesions that were visualized both with MRI and contrast-enhanced ultrasound (van den Bos W et al., Eur Radiol, 2015, 1-9). Focal laser ablation (FLA), or laser interstitial thermal therapy, relies on localized heating of the prostate via a fiber-coupled infrared laser (Lindner U et al., The Journal of Urology, 2009, 182(4):1371-1377). Unlike HIFU, FLA relies on coagulative necrosis to remove tissue while avoiding cavitation, carbonization, or vaporization (McNichols R J et al., International Journal of Hyperthermia, 2004, 20(1):45-56). Unlike HIFU or IRE, FLA provides the opportunity for treatment without general anesthesia.

The purpose of the following study was to gather safety and feasibility data and to explore the potential to simplify FLA. The primary endpoint in this Phase I trial was absence of any grade 3 adverse event (CTCAE, v4.03). Exploratory endpoints were changes in sexual and urinary function compared to baseline, as well as radiologic and histologic changes. To date, FLA has almost exclusively been performed within an

MRI tube (in-bore) because of direct image-guidance and the potential utility of MR-thermometry (MRT) for intra-prostatic temperature monitoring during treatment (Oto A et al., Radiology, 2013, 267(3):932-940). In the following study, MR-compatible thermal probes were placed at various locations within the patient's prostate before FLA. The study design allowed simultaneous comparison of MRT and direct thermal recordings during FLA (Oto A et al., Radiology, 2013, 267(3):932-940).

The materials and methods are now described.

### Patients

Patients in this trial were eight men ages 58-72 years old with clinical stage≤T2b CaP and Gleason Score (GS)≤3+4=7. All eight were diagnosed by MR/US fusion biopsy, incorporating both targeted and systematic sampling (Sonn GA et al., The Journal of Urology, 2013, 189(1):86-92), which showed CaP within a single MR-visible lesion and no GS>6 in the prostate. The men were selected per entry criteria from those undergoing fusion biopsy in a cohort described elsewhere (Sonn GA et al., The Journal of Urology, 2013, 189(1):86-92). 3T MRI using a body coil was acquired and interpreted using both PI-RADS and a 5-point grading system devised in-house (Sonn G A et al., The Journal of Urology, 2013, 189(1):86-92). FLA was performed within 6 months of diagnosis. Patient characteristics are given in FIG. 25.

### Procedure Planning

MR-enhancing index regions of interest (ROI) with biopsy-confirmed cancer were targeted using FLA. ROI characteristics were determined by 3D segmentation of the MRI. Fiber locations and desired margins were planned in advance using custom software developed using MATLAB and C++according to each patient's ROI geometry and location within the prostate. Prior work with MRI-histopathology correlation indicates that MRI systematically underestimates true tumor volume by up to 1.5 cm (Priester A et al., Int Symp Focal Therapy Imag 2014, Pasadena, Calif., Aug 21-23, PP-24). This margin was then further refined by using prior biopsy information, i.e. 3D locations of positive and negative cores. Based on preliminary data obtained during a sizeable in-bore experience, it was estimated that a 3 minute laser activation at 12-15 W would create a zone of coagulation necrosis extending radially approximately 1 cm around the laser tip.

### Treatment protocol

Prior to positioning patients in the MRI tube, all subjects received a cleansing saline enema and antibiotics: 5 days of oral ciprofloxacin starting one day before FLA and intramuscular ceftriaxone at the time of FLA. A peri-prostatic block of 1% lidocaine and 0.5% bupivacaine was administered under transrectal ultrasound guidance. Local anesthesia was supplemented by intravenous doses of versed and fentanyl (conscious sedation) as needed. Prior to FLA, two to three MR-compatible fluoroptic temperature probes (STB, LumaSense, Santa Clara, Calif.) were advanced into the prostate through brachytherapy applicators (Flexi-needle, Best Medical, Springfield, Va.) placed transperineally under ultrasound guidance. The temperature probes were placed for assessment of intra-prostatic thermal changes, independent of MRT. For each patient, at least one probe was inserted into the posterior prostate near the rectal wall.

Patients were then transported to the MR suite and placed in prone position within the gantry. A 1.5 T scanner with trans-abdominal coil (Avant, Siemens) was used. A transrectal prostate needle guide (DynaTRIM, Invivo Corp., Gainsville, Fla.) was used to place laser fibers in the prostate. The Visualase system (Biotex/Medtronic, Houston, Tex.), consisting of a 15 W, 980 nm laser, cooling pump, and MR thermometry analysis workstation, was used for all treatments. The system incorporates a 600 μm laser fiber within a dual lumen catheter that circulates saline to actively cool the fiber. Confirmation of laser position was made with T2-weighted MRI prior to application of laser energy.

During treatment, intra-prostatic temperature was continuously monitored and recorded by MRT every 6 seconds and by the thermal probes in real-time. A typical example of the spatial relationship of the laser fiber and the probes within the prostate during treatment is shown in FIG. 11. Position of the fiber and probes were periodically reconfirmed by MRI scanning.

Prior to each laser treatment, a test dose of 6-8 W was used to localize the laser fiber under MRT. Laser power and cooling flow rates were manually adjusted by the performing physician according to MRT feedback. Multiple laser applications per fiber insertion were performed as needed for complete lesion treatment by advancing or withdrawing the fiber in the line of insertion prior to retreatment.

Using laser software and MRT, the laser tip and rectal wall temperatures were monitored to ensure temperatures did not exceed 90° C. and 42° C., respectively. In case of temperature exceeding monitor threshold, the laser application was ceased automatically. The Visualase software provides processing of MRT images and indication of treatment progress (McNichols R J et al., International Journal of Hyperthermia, 2004, 20(1):45-56; Lee T et al., Reviews in Urology, 2014, 16(2):55).

### Follow-Up Evaluations

Immediately after treatment, mpMRI was obtained and evaluated. Dynamic contrast-enhanced MRI was used to confirm the treatment zone and to compare it to the planned treatment zone and MRT map. Patients were monitored in a recovery room and after voiding, and all were discharged within a few hours. Discharge medications included a quinolone antibiotic and oral non-narcotic analgesics.

Digital rectal exam (DRE), urinalysis, post-void residual volume, International Prostate Symptom Score (IPSS), Sexual Health Inventory for Men (SHIM), and Prostate Specific Antigen (PSA) were obtained at clinic visits 1 week, 1 month, 3 months, and 6 months after FLA. Repeat mpMRI and targeted biopsy of the prostate were performed six months after treatment using MR/US fusion as before treatment (Artemis, Eigen, Grass Valley, Calif.). Targeted biopsy cores of the treated area and margin were sampled in addition to systematic cores on the side of treatment. 3T MRI was performed at baseline and 6 month follow-up and interpreted using PI-RADS v2 scoring (Barentsz J O et al., Eur Radiol, 2012, 22:746) in addition to scoring criteria developed by UCLA (Sonn G A et al., The Journal of Urology, 2013, 189(1):86-92; Natarajan S et al., Urologic Oncology: Seminars and Original Investigations, 2011, 29(3):334).

The results are now described.

During each procedure, the laser fiber was reintroduced an average of 3 times, involving an average of 7 applications per patient at a power of 11-14 W. The aim was to perform each application as long as the MRT feedback safety mechanism would allow. Mean procedure time was 292 minutes, including patient preparation, thermal probe insertion, laser treatment, and post-treatment imaging. Actual time within the MRI scanner averaged 223 minutes (range, 169-267 minutes).

Median prostate volume decreased from 35.5 cc to 32.5 cc (MRI) after 6 months (p=0.03, Wilcoxon signed-rank test, FIG. 26). Median PSA at baseline was 7.45 ng/mL and decreased significantly to 3.3 ng/mL after 1 month, a change that persisted at six months (p<0.01, Wilcoxon signed-rank test). In five of the eight men, PSA at six months decreased to less than half the value at screening. Percent free PSA increased significantly from 7.5% to 14% (p =0.047, Wilcoxon signed-rank test). Median PSA density dropped from 0.22 to 0.08 ng/mL (p =0.055, Wilcoxon signed-rank test). PSA results for all eight men are given in FIG. 27.

### Adverse Events

23 Grade 1 and 7 Grade 2 adverse events (CTCAE, v4.03) were recorded over a 9-month period, all of which resolved spontaneously. Symptoms that were most prevalent were hematuria (12), hematospermia (4), and blood in stools (1). All Grade 2 events resolved within 8 days of assessment. All patients left the hospital within 6 hours, and none required narcotic analgesia for pain relief after the treatment.

### Health-Related Quality of Life Measures

IPSS and SHIM were collected on all eight men at screening, and at 1 week, 1 month, 3 months, and 6 months. Median IPSS was 4 at screening, and decreased to 3.5 at 6 months. Median SHIM was 19.5 at screening, and increased to 20 at 6 months. No statistically significant changes in either health-related quality of life metric were observed after six months (p=0.37, p=0.78, respectively). No urinary incontinence, erectile dysfunction, or change in ejaculation was reported by any patient.

### Temperature data

MRT data were successfully collected in all patients, but these data were highly sensitive to patient motion (FIG. 28). Data from fluoroptic thermal probes were recorded in six of eight patients, the first two being unsatisfactory technically. In these six patients, mean temperatures were below 40° C. in all intra-prostatic locations outside of the treatment zone (FIG. 29A, FIG. 29B).

### MRI Changes

For each patient, multi-parametric MRI (mpMRI) was acquired prior to diagnosis, just prior to intervention, immediately post-FLA, and again at 6 months. Tissue volume and changes on perfusion were noted, and summarized in FIG. 26. There were no consistent changes in T2 or DWI; DCE alone was used to determine an immediate post-treatment effect (Oto A et al., Radiology, 2013, 267(3):932-940). In each patient, the region of limited perfusion was within the treated area, away from critical structures, with a median volume of 3 cc (FIG. 30). Morphologic changes in the gland, including swelling during treatment, and significant shrinkage post-treatment (p=0.03, Wilcoxon signed-rank test) confounded localization of the treatment area. In general, the treatment zone, as denoted by immediate post-FLA DCE, was no longer apparent at 6 months.

### Histologic Changes

Follow-up targeted biopsy was performed six months after FLA using MR/US fusion (Filson C P et al., Calif.: A Cancer Journal for Clinicians, 2015, 65:265). Biopsies were targeted at the treatment zone/original cancer focus, margin around treatment zone, and systematic biopsies on the treated side. A mean of 15 cores (range: 13-17) were obtained from each patient. Biopsies revealed no evidence of any safety concerns (i.e., no infectious, traumatic, or neoplastic adverse changes were seen). The commonest treatment-related finding was a focal area of fibrosis, often interspersed with the presence of hemosiderin-laden macrophages, indicating resorption of old hemorrhage (FIG. 31).

In five of eight men, no cancer was found in the treated region. In patients 3, 7, and 8, CaP was found in the treated area (7.5 mm GS 3+4, 2.5 mm GS 3+4, 1 mm GS 6, all lengths refer to maximum cancer core length in millimeters). In tissue outside, but adjacent to the treatment zone, six patients had persistent tumor (1.4 mm GS 4+4, 5.5 mm GS 3+4, 7.5 mm GS 3+4, 2.5 mm GS 6, 0.5 mm GS 6, 8 mm GS 6). One patient was found to have tumor on systematic biopsy remote from the treatment zone (3 mm GS 3+4). No CaP was found upon biopsy of patient 6.

The trend toward minimally invasive treatments, accelerating over the past several decades, will likely have a substantial impact on prostate cancer (CaP) care. According to estimates from the National Cancer Institute, focal therapies may encompass up to 25% of all CaP treatments in the near future (Mariotto A B et al., Journal of the National Cancer Institute, 2011, 103:699). Focal therapy offers the hope of cancer control with reduced treatment-related morbidity, for a subset of the CaP population not yet defined. However, data on safety and efficacy of the new interventions are sparse, and clinical trials utilizing image-guided treatment are few (Klotz L et al., Nature Reviews Clinical Oncology, 2014, 11(6):324-334).

In the present study, the potential for focal laser ablation (FLA) of the prostate was advanced in several ways. First, confirming earlier studies, FLA of the prostate was shown to be safe in men with intermediate-risk CaP, without serious adverse events or change in urinary or sexual function (Oto A et al., Radiology, 2013, 267(3):932-940; Lindner U et al., The Journal of Urology, 2009, 182(4):1371-1377; Lindner U et al., Journal of Endourology, 2010, 24(5):791-797; Lindner U et al., The Journal of Urology, 2013, 4(189):e227-e228). The transrectal approach proved to be feasible. Second, the addition of secondary safety monitors confirms that laser temperatures are well confined to the intended treatment zone, even during a three-minute activation at nearly 14 W. Third, the comprehensive biopsy follow-up results in this study indicate that larger margins than previously thought may be necessary for effective focal therapy. LeNobin et al. suggest that a margin of one centimeter around the MRI target may be required for complete tumor ablation Le Nobin J et al., The Journal of Urology, 2015, 194:364).

The cancers treated in the present study were intermediate, not low risk (NCCN). In the Oto study, cancers treated were small spots of Gleason 3+3 =6 in 7 of 8 patients (Oto A et al., Radiology, 2013, 267(3):932-940). In the present study, 7 of 8 men had Gleason Scores of 3+4 =7 and median maximum core length of over 4 mm. Patient selection here was thus in keeping with current consensus recommendations to treat men of intermediate risk (Donaldson I A et al., European Urology, 2015, 67(4):771-777). At 6 months of follow-up, cancer was undetectable upon comprehensive biopsy of the original cancer-bearing focus in 5 of the 8 patients, suggesting the potential for effective FLA in intermediate-risk individuals.

A curative success-rate for FLA is difficult to determine from these initial studies. The present in-bore study was a preliminary experience with FLA and safety was a primary concern, making laser deployments conservative. A larger margin or more aggressive treatment parameters that previously employed may be effective in eliminating all CaP in an intermediate risk population.

### Example 5: Office-Based Focal Laser Ablation

Focal laser ablation (FLA) has been used to safely treat prostate cancer (CaP) under real-time MRI guidance but is cumbersome, lengthy, resource-intensive, and approached as a radiological procedure. The following study used an extensive targeted biopsy experience to provide the basis for performing FLA in a urology clinic under Mill/ultrasound (MRI/US) fusion guidance.

Four male patients having biopsy-confirmed intermediate risk (Gleason 3+4) CaP in a single MR-visible lesion participated in the study. FLA was performed transrectally under MRI/US fusion guidance (Artemis) using a 980 nm, 15 W water-cooled laser (Visualase). A peri-prostatic block was supplemented by intravenous midazolam. Custom software was created to monitor treatment temperatures in real-time using four interstitial thermal probes. At least one probe was placed adjacent to the rectal wall to assess safety, and one was placed parallel to the laser fiber to monitor the temperature at the laser tip. Multi-parametric MRI, including dynamic contrast enhancement (DCE) was performed following treatment.

FLA was successfully performed in four patients without incident or serious adverse events. In each patient, two to three laser applications of 3 minutes each were used. Total procedure time, from initial ultrasound scan to probe removal, averaged 93 minutes (range, 91-100 minutes), and patients were discharged within 4 hours of treatment. Ablation volumes, seen on post-treatment DCE MRI (FIG. 32B), were 3.8 cc on average (range, 2.5-4.7 cc). The thermal probe adjacent to the laser tip recorded a temperature exceeding 60° C. in every case. The rectal wall temperature did not exceed 42° C. in any patient.

FLA in a urologic clinic setting, under MRI/US fusion guidance, is feasible and was safely performed in four men. Thermal probe recordings proved reliable and convenient, demonstrating the ability to replace MRI thermometry for FLA. A potential for focal therapy of prostate cancer to remain a urological procedure was demonstrated.

### Example 6: 3D-Printed Patient-Specific Prostate Molds to Define MRI-Whole Organ Relationships in Prostate Cancer

Prostates from 65 men (median 61 years old, range 44-79 years old) who received radical prostatectomy were precisely sectioned using patient-specific 3D-printed molds. These molds were generated from pre-operative mpMRI contours (FIG. 33A), where each slice corresponded to an MR image plane (FIG. 33B). The tumors were delineated on whole mount slides (FIG. 33C), digitally reconstructed (FIG. 33D), and matched to corresponding MRI lesions (UCLA grades 3-5) using MATLAB software. All patients were previously untreated, and mean prostate volume was 40 cc (range 19-110 cc).

91 MRI lesions and 126 actual tumors were spatially correlated in the 65 men, with predictive accuracy summarized in FIG. 35 on a per-tumor basis. Clinically significant prostate cancer (csCaP), i.e., any Gleason Sum (GS)≥7 or any GS 6≥0.5 cc, was found in 88% of patients. In 30% of patients, at least one csCaP tumor was undetected on MRI; average volume of these tumors was 1.9 cc (range 0.5-6.9 cc). For detection of all csCaP, MRI sensitivity was 76% and specificity was 64%. Furthermore, MRI sensitivity and specificity for csCaP increased with mpMRI suspicion score (FIG. 34).

Patient-specific 3D-printed molds enable accurate MR-histology correlation and rigorous evaluation of the predictive utility of mpMRI. The majority of tumors were detected on MRI, and most undetected tumors were small-volume and/or Gleason 3+3. However, at least one clinically significant tumor region was missed on mpMRI in 30% of patients.

### Example 7: Informing Focal Therapy Margins through MRI-Pathology Correlation

Multi-parametric MRI (mpMRI) is a robust method for imaging prostate cancer (CaP) and guiding targeted interventions. The following study investigates the spatial relationship between MRI-visible regions of interest (ROIs) and areas of known CaP and to characterize the treatment margins necessary for effective focal therapy.

Prior to radical prostatectomy, 65 men underwent mpMRI, from which a radiologist contoured the prostate capsule and regions suspicious for CaP. A custom mold was then 3D-printed from the patients' MRI and used for precise sectioning of the surgical specimen. This mold facilitated accurate matching of the delineated slides (FIG. 36A) with preoperative mpMRI (FIG. 36B). All tumors found on pathology were digitally reconstructed in 3D and matched to corresponding MRI targets (n=71). The geometric features of all surfaces and the maximum distance between each MRI target and matched tumor were determined using custom software.

Spatial features of ROIs and tumors are summarized in FIG. 37. The mean volume and longest axis of the prostate capsule corresponded closely with MRI measurements, yet the mean volume of CaP was 2.7 times greater than the ROI predictions. The mean longest axis on MRI was found to be 16.8 mm, whereas the mean longest axis on pathology was 27.5 mm. Due to tumor asymmetry, CaP extended an average of 15 mm beyond the ROI along at least one axis (FIG. 36C). Retrospectively, only a minority of these tumor extensions was identifiable on MRI.

MRI underestimated CaP volume by a factor of 2.7 (0.9 cc on MRI vs. 2.4 cc on pathology). Using MRI targeting alone, effective focal therapy would need to include substantial margins around the ROI (median 15 mm). In practice, this margin could be reduced using tracked biopsy information or better imaging to characterize tumor asymmetry.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-12. (canceled)

13. A multi-channel needle guide device comprising:
an elongate body;
a first channel having a first channel centerline;
at least one auxiliary channel having an auxiliary channel centerline; and
a plurality of attachment clips.

14. The device of claim 13, further comprising a locking member selected from the group consisting of: a screw, a clamp, a bolt, and a pin.

15. The device of claim 13, wherein the plurality of attachment clips comprises tabs, hooks, or slots to secure the multi-channel needle guide device to the body of an ultrasound probe.

16. The device of claim 13, wherein the first channel has a lumen sized suitably for a biopsy needle, catheter, laser fiber, or trocar to pass therethrough.

17. The device of claim 13, wherein the at least one auxiliary channel has a lumen sized suitably for a thermal sensor, an optical sensor, or a multi-modal sensor to pass therethrough.

18. The device of claim 13, wherein the first channel centerline and the auxiliary channel centerline are spaced between 1 and 20 mm apart.

19. The device of claim 13, further comprising at least one additional auxiliary channel.

20-29. (canceled)

30. A multi-modal sensor probe comprising:
an elongate thermal sensor;
at least one optical fiber positioned adjacent and parallel to the thermal sensor;
an angled light-directing surface positioned at one end of the at least one optical fiber; and
a housing encasing the thermal sensor, the at least one optical fiber, and the light-directing surface.

31. A multi-modal sensor probe comprising:
at least one optical fiber, each optical fiber adjacent and parallel to each other;
a temperature-sensitive element-positioned on each optical fiber; and
a housing encasing the at least one optical fiber and the temperature-sensitive elements.

32. The multi-modal sensor probe of claim 31 wherein the temperature-sensitive element is phosphor.

33. The multi-modal sensor probe of claim 30, wherein the thermal sensor is a fluoroptic thermal probe.

34. The multi-modal sensor probe of claim 30, having an outer diameter of about 1.5 mm.

35. The multi-modal sensor probe of claim 30, wherein the probe is constructed from an optically transparent material that is thermally stable in the range of about 0° to 120° C.

36. The multi-modal sensor probe of claim 31, having an outer diameter of about 1.5 mm.

37. The multi-modal sensor probe of claim 31, wherein the probe is constructed from an optically transparent material that is thermally stable in the range of about 0° to 120° C.

38. A method of cancer margin determination in soft tissue comprising the steps of:
acquiring at least one MRI image of a region of interest having at least one MRI-visible lesion;
generating a 3D model of the at least one MRI-visible lesion from the at least one MRI image;
acquiring at least one biopsy core from the tissue surrounding the MRI-visible lesion;
categorizing the at least one biopsy core as a cancer-containing positive node, a cancer-absent negative node, or a neutral node having an indeterminate cancer presence;
at least partially expanding the 3D model of the at least one MRI-visible lesion to encompass any locations of positive nodes comprising cancerous tissue to generate a minimum treatment volume (MTV) 3D model;
at least partially expanding the MTV 3D model to cover any potentially cancerous tissue; and
at least partially contracting the MTV 3D model to exclude any locations of negative nodes to generate an Optimized Margin 3D model.

39. The method of claim 38, wherein the MTV 3D model is at least partially expanded to encompass the location of neutral nodes.

40. The method of claim 38, wherein the MTV 3D model is isotropically expanded by 1 cm in all directions.

41. The method of claim 38, wherein the MTV 3D model is at least partially expanded to encompass regions that appear to be cancer harboring based on medical image data.

42. The method of claim 38, wherein the MTV 3D model is at least partially expanded to encompass cancer-containing regions based on statistical analysis of a population of previous biopsies, a population of previously treated patients, or both.

43. A system for focal laser therapy of soft tissue comprising:
a laser;
at least one thermal sensor;
a needle guide;
an ultrasound probe;
3D scanning and location tracking assembly; and
a computer platform.

44. The system of claim 43, further comprising at least one optical sensor.

45. The system of claim 43, further comprising at least one multi-modal sensor having at least one thermal sensing element and at least one optical sensing element.

46. The system of claim 43, wherein the laser comprises a laser fiber, a coolant, a dual lumen catheter, a cooling pump, a flow sensor, and a flow controller.

47. The system of claim 46, wherein the laser fiber is capable of emitting between 5 and 50 W of light.

48. The system of claim 46, wherein the coolant is an inert solution of water or saline.

49. The system of claim 46, wherein the coolant is room temperature or below room temperature.

50. A method of focal laser therapy of soft tissue, comprising the steps of:
obtaining a real-time 3D ultrasound model of a patient's region of interest to be treated;
overlaying at least one cancer margin 3D model over the real-time 3D ultrasound model;
generating at least one expected damage model, wherein the at least one expected damage model at least partially overlaps the at least one cancer margin 3D model;
calculating at least one laser fiber location in the patient's region of interest and at least one ablation setting to fit the at least one expected damage model, wherein the at least one ablation setting comprises a laser power output, a laser exposure duration, a laser exposure rate, and a coolant flow rate;
calculating at least one sensor location in the patient's region of interest;
inserting a laser fiber into the at least one laser fiber location and at least one sensor into the at least one sensor location;
ablating the region of interest according to the at least one ablation setting; and
monitoring treatment progression by modelling the extent of ablated tissue

51. The method of claim 50, wherein the at least one cancer margin 3D model comprises a MRI-visible lesion 3D model, a MTV 3D model, an Optimized Margin 3D model, and biopsy core location.

52. The method of claim 50, wherein the expected damage model comprises three nested ellipsoids, the smallest ellipsoid representing minimum expected damage (minED), the medium ellipsoid representing average expected damage (aveED), and the largest ellipsoid representing maximum expected damage (maxED)

53. The method of claim 52, wherein the minED of the expected damage model encapsulates the entirety of the MTV 3D model.

54. The method of claim 50, wherein the at least one sensor comprises at least one thermal sensor, at least one optical sensor, at least one multi-modal sensor, or any combination thereof

55. The method of claim 50, wherein the ablation settings are limited from generating a temperature higher than 95° C.

56. The method of claim 50, wherein the extent of tissue damage is modelled by measuring the temperature of tissue adjacent to the region of interest being treated.

57. The method of claim 50, wherein the extent of tissue damage is modelled by measuring the rate of tissue cooling immediately after executing the at least one ablation setting.

58. The method of claim 50, wherein the extent of tissue damage is modeled by ultrasound measurements of tissue temperature change, mechanical property change, vascularity change, or appearance change with a contrast agent.

59. The method of claim 50, wherein the extent of tissue damage is modelled by quantifying the level of thermally induced alterations in tissue optical properties.
